(19) United States
(12) Patent Application Publication
(10) Pub. No.: US 2010/0004293 A1
(43) Pub. Date: Jan. 7, 2010

(54) CRYSTALLINE FORMS OF ARYL-SUBSTITUTED PYRAZOLE-AMIDE COMPOUNDS

(75) Inventor: Mary F. Malley, Lawrenceville, NJ (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(73) Assignee: Bristol-Myers Squibb Company

(21) Appl. No.: 12/518,490

(22) PCT Filed: Dec. 19, 2007
(86) PCT No.: PCT/US2007/088077

§ 371 Date: Aug. 13, 2009

Related U.S. Application Data
(60) Provisional application No. 60/875,892, filed on Dec. 20, 2006.

(51) Int. Cl. A61K 31/4439; C07D 401/04; A61P 29/00

(52) U.S. Cl. 514/341; 546/275.4

(57) ABSTRACT

The present invention provides novel crystals of Compound I, pharmaceutical compositions containing such novel form and a method of treating p38 kinase associated conditions, including rheumatoid arthritis, using such novel form.

This application claims priority to U.S. Provisional Patent Application Ser. No. 60/875,892, filed Dec. 20, 2006, which is hereby incorporated by reference in its entirety.

### FIELD OF THE INVENTION

The present invention generally relates to crystalline forms of N-(5-(cyclopropylcarbamoyl)-2-methylphenyl)-5-methyl-1-(3-(trifluoromethyl)pyridin-2-yl)-1H-pyrazole-4-carboxamide. The present invention also generally relates to a pharmaceutical composition comprising said crystalline form, as well of methods of using the crystalline form in the treatment of inflammatory diseases, and methods for obtaining such crystalline forms.

### BACKGROUND OF THE INVENTION

U.S. Patent Application Publication No. US-2005-0159424-A1 discloses the compound N-(5-(cyclopropylcarbamoyl)-2-methylphenyl)-5-methyl-1-(3-(trifluoromethyl)pyridin-2-yl)-1H-pyrazole-4-carboxamide having the structure of formula I:

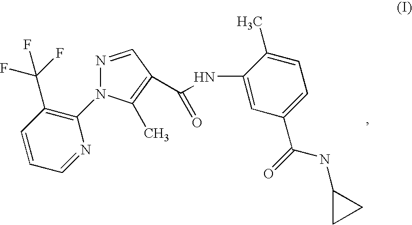

and pharmaceutically-acceptable salts, prodrugs, solvates, isomers, and/or hydrates thereof, which are advantageous as inhibitors of p38 kinase and may be used for treating p38 kinase-associated conditions, including rheumatoid arthritis. The compound of formula I is referred to herein as “Compound I”.

Processes to prepare Compound I and methods of treatment employing Compound I are also disclosed in U.S. Patent Publication No. 2005/0159424 A1. This reference is assigned to the present assignee and is incorporated herein by reference in its entirety. Specifically, U.S. Patent Publication No. 2005/0159424 A1 further discloses that Compound I may be prepared using the reaction sequences disclosed in Schemes 1-8 therein, which are incorporated by reference herein, particularly as to the methods of preparation.

### BRIEF DESCRIPTION OF THE INVENTION

In accordance with one aspect of the invention, novel crystalline form of Compound I and a process for selectively preparing such a novel crystalline form of Compound I are provided.

### BRIEF DESCRIPTION OF THE DRAWINGS

FIG. 1 shows observed (experimental at 22° C.) and simulated (calculated at 22° C.) powder x-ray diffraction patterns (CuKα λ=1.5418 Å) of the N-2 crystalline form of Compound I.

FIG. 2 shows a differential scanning calorimetry (DSC) thermogram of the N-2 crystalline form of Compound I.

FIG. 3 shows a thermogravimetric analysis (TGA) curve of the N-2 crystalline form of Compound I.

FIG. 4 shows observed (experimental at 22° C.) and simulated (calculated at 22° C.) powder x-ray diffraction patterns (CuKα λ=1.5418 Å) of the H-1 crystalline form of Compound I.

FIG. 5 shows a differential scanning calorimetry (DSC) thermogram of the H-1 crystalline form of Compound I.

FIG. 6 shows a thermogravimetric analysis (TGA) curve of the H-1 crystalline form of Compound I.

FIG. 7 shows observed (experimental at 22° C.) and simulated (calculated at 22° C.) powder x-ray diffraction patterns (CuKα λ=1.5418 Å) of the N-7 crystalline form of Compound I.

FIG. 8 shows a differential scanning calorimetry (DSC) thermogram of the N-7 crystalline form of Compound I.

FIG. 9 shows a thermogravimetric analysis (TGA) curve of the N-7 crystalline form of Compound I.

FIG. 10 shows observed (experimental at 22° C.) and simulated (calculated at 22° C.) powder x-ray diffraction patterns (CuKα λ=1.5418 Å) of the N-5 crystalline form of Compound I.

FIG. 11 shows a differential scanning calorimetry (DSC) thermogram of the N-5 crystalline form of Compound I.

FIG. 12 shows a thermogravimetric analysis (TGA) curve of the N-5 crystalline form of Compound I.

FIG. 13 shows observed (experimental at 22° C.) and simulated (calculated at 30° C.) powder x-ray diffraction patterns (CuKα λ=1.5418 Å) of the N-6 crystalline form of Compound I.

FIG. 14 shows a differential scanning calorimetry (DSC) thermogram of the N-6 crystalline form of Compound I.

FIG. 15 shows a thermogravimetric analysis (TGA) curve of the N-6 crystalline form of Compound I.

FIG. 16 shows observed powder x-ray diffraction patterns (CuKα λ=1.5418 Å at T=22° C.) of the P-14 crystalline form of Compound I.

FIG. 17 shows a differential scanning calorimetry (DSC) thermogram of the P-14 crystalline form of Compound I.

FIG. 18 shows a thermogravimetric analysis (TGA) curve of the P-14 crystalline form of Compound I.

FIG. 19 shows simulated (calculated at −50° C.) powder x-ray diffraction patterns (CuKα λ=1.5418 Å) from the form-3 family of solvates (EA.5-3, DC-3 (calculated at −50 degrees C.) and AN-3 (calculated at −70 degrees C.) of Compound I.

FIG. 20 shows simulated (calculated at −50° C.) powder x-ray diffraction patterns from form SA-9 of Compound I.

FIG. 21 shows observed (slurry, rt) and calculated (−60° C.) PXRD of Form SC-13 (2 THF, 1 H₂O).

FIG. 22 shows simulated and observed PXRD of form SD-14 and sPXRD of form H-1.

### DEFINITIONS

The names used herein to characterize a specific form, e.g., “N-2”, should not be considered limiting with respect to any other substance possessing similar or identical physical and chemical characteristics, but rather it should be understood that these designations are mere identifiers that should be interpreted according to the characterization information also presented herein.

The present invention provides, at least in part, a crystalline form of Compound I as a novel material, in particular in a pharmaceutically acceptable form. The term “pharmaceutically acceptable,” as used herein, refers to those compounds, materials, compositions, and/or dosage forms which are, within the scope of sound medical judgment, suitable for contact with the tissues of human beings and animals without excessive toxicity, irritation, allergic response, or other complications commensurate with a reasonable benefit/risk ratio. In certain preferred embodiments, crystalline forms of Compound I are in substantially pure form.

As used herein “polymorph” refers to crystalline forms having the same chemical composition but different spatial arrangements of the molecules, atoms, and/or ions forming the crystal.

As used herein “solvate” refers to a crystalline form of a molecule, atom, and/or ions that further contains molecules of a solvent or solvents incorporated into the crystalline structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate.

As used herein “amorphous” refers to a solid form of a molecule, atom, and/or ions that is not crystalline. An amorphous solid does not display a definitive X-ray diffraction pattern.

As used herein, “substantially pure,” when used in reference to a crystalline form, means a compound having a purity greater than 90 weight %, including greater than 90, 91, 92, 93, 94, 95, 96, 97, 98 and 99 weight %, and also including equal to about 100 weight % of Compound I, based on the weight of the compound. The remaining material comprises other form(s) of the compound, and/or reaction impurities and/or processing impurities arising from its preparation. For example, a crystalline form of Compound I may be deemed substantially pure in that it has a purity greater than 90 weight %, as measured by means that are at this time known and generally accepted in the art, where the remaining less than 10 weight % of material comprises, for example, other form(s) of Compound I and/or reaction impurities and/or processing impurities.

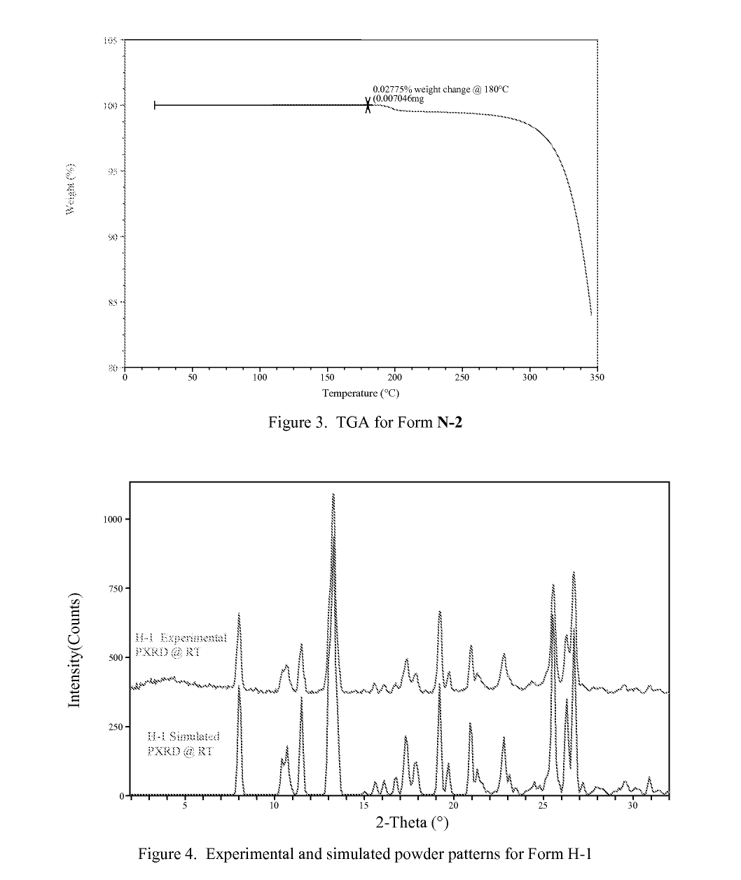

The term “negligible weight loss,” as employed herein, as characterized by TGA indicates the presence of a neat (non-solvated) crystal form. From a quantitative view, this term means the crystalline form as defined in, for example, Claim 2 is characterized by a thermal gravimetric analysis curve in accordance with that shown, for example, in FIG. 3, having a weight loss≦0.028% at about 180° C.

The term “negligible % water uptake,” as employed herein, as characterized by moisture-sorption isotherm indicates that the form tested is non-hygroscopic.

### DETAILED DESCRIPTION OF THE INVENTION

The invention relates to a novel crystalline form of Compound I, which is described and characterized herein.

In particular, the present invention is for the N-2 crystalline form of Compound I.

The monohydrate H-1 is unstable under reduced humidity and converts topotactically upon heating (90° C., 30 m) to a neat form, T1H1 (N-7), with ˜15% (1.6 Å) contraction of the crystallographic a axis.

In one embodiment, neat form N-2 was crystallized from BuOAc, iPrOAc and acetone, while other neat forms N-5, N-6 and N-7 have been obtained from the melt.

N-2 is the most stable polymorphic (neat) form at 25° C. and 50° C. Based on slurry conversion studies at 25 and 50° C., and melting data: N-2:N-5 and N-2:N-6 are enantiotropic with a transition temperature between 50 and 204° C.; N-5:N-6 are monotropes with N-5 being the more stable; N-7 is monotropic with N-2, N-5 and N-6 and therefore less stable at all temperatures below 192° C. The high temperature dehydration/conversion of H-1 to the metrically similar N-7 structure, rather than the more stable N-2, is presumably related to topotactic nucleation.

Slurry conversion studies in butanol/water mixtures show that at RH≦16%, N-2 is the more stable form, and at RH≧30%, H-1 is the more stable form. The water sorption data for dehydrated H-1 (presumably N-7) show that in the ascending RH run, the sample partially rehydrates to H-1 between 75 and 95% RH. In the descending run, H-1 is partially dehydrated (presumably to N-7) at 25% RH. The available data establish the following RH range for equilibrium N-2/H-1 conversion: 25%<RHeq<=30%. Wetcakes subjected to RH<30% are subject to dehydration, kinetic factors notwithstanding.

In addition to the monohydrate, Compound I also forms solvates with a large number of organic solvents. The type 3 family of solvates (represented by an EtOAc solvate, form EA.5-3; a PrOAc solvate, form PA.5-3; a MeCN solvate, form AN-3; and a CH₂CL₂ solvate, DC-3) have a large hydrophobic clathrate channel (V˜215 Å³) parallel to the crystallographic a repeat. A (1:1) methanolate, form M-4, proved to be relatively stable; the solvent site is fully occupied when the structure is determined at −50° C. and is still 50% occupied when the structure was re-determined at +82° C. The single crystal structures of an ethanol solvate (E-8), an isopropanol solvate (IPA-10), a second acetonitrile form (AN-11) and three mixed solvates (SA-9, SB-12, SC-13) with THF and water have been determined.

N-2 and H-1 are chemically and physically stable in the solid-state when stored at 50° C./75% RH open and closed for at least 4 weeks and when exposed to HIL for at least 1 week. The aqueous solubility of N-2 is ≧28 μg/mL, while H-1 has a solubility of 7 μg/mL. A monkey PK crossover study (n=4) showed that this ˜4× solubility ratio results in approximately a 2-fold greater exposure for the N-2 relative to H-1. Drying studies for H-1 showed that when the dryer was maintained at 40° C. at RH 2.4-3.4%, H-1 converted to N-7, whereas no conversion was observed at 20-30% RH. Based on the equilibrium RH studies, N-2 could undergo hydrate formation during handling, formulation or storage. However, studies of N-2 stored for 3 weeks or more at elevated temperature and elevated humidity (e.g., 50° C./75% RH) and in the presence of common filler excipients showed no conversion.

Samples of the crystalline forms may be provided with substantially pure phase homogeneity, indicating the presence of a dominant amount of a single crystalline form and optionally minor amounts of one or more other crystalline forms. The presence of more than one crystalline form in a sample may be determined by techniques such as powder X-ray diffraction (PXRD) or solid state nuclear magnetic resonance spectroscopy (SSNMR). For example, the presence of extra peaks in the comparison of an experimentally measured PXRD pattern with a simulated PXRD pattern may indicate more than one crystalline form in the sample. The simulated PXRD may be calculated from single crystal X-ray data. See Smith, D. K., “A FORTRAN Program for Calculating X-Ray Powder Diffraction Patterns,” Lawrence Radiation Laboratory, Livermore, Calif., UCRL-7196 (April 1963).

Preferably, the crystalline form has substantially pure phase homogeneity as indicated by less than 10%, preferably less than 5%, and more preferably less than 2%, of the total peak area in the experimentally measured PXRD pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

Procedures for the preparation of crystalline forms are known in the art. The crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture.

The forms may be characterized and distinguished using single crystal X-ray diffraction, which is based on unit cell measurements of a single crystal of a form at a fixed analytical temperature. A detailed description of unit cells is provided in Stout & Jensen, X-Ray Structure Determination: A Practical Guide, Macmillan Co., New York (1968), Chapter 3, which is herein incorporated by reference as to unit cells and their use. Alternatively, the unique arrangement of atoms in spatial relation within the crystalline lattice may be characterized according to the observed fractional atomic coordinates. Another means of characterizing the crystalline structure is by powder X-ray diffraction analysis in which the experimental or observed diffraction profile is compared to a simulated profile representing pure powder material, both run at the same analytical temperature, and measurements for the subject form characterized as a series of 2 theta (“2Θ”) values.

Other means of characterizing the form may be used, such as solid state nuclear magnetic resonance (SSNMR), differential scanning calorimetry and thermogravimetric analysis. These parameters may also be used in combination to characterize the subject form. Such methods are known to those skilled in the art.

In one embodiment of the invention, a crystalline form of Compound I is provided in substantially pure form. This crystalline form of Compound I may be employed in pharmaceutical compositions which may optionally include one or more other components selected, for example, from the group consisting of excipients, carriers, and one of other active pharmaceutical ingredients or active chemical entities of different molecular structures.

Preferably, the crystalline form has substantially pure phase homogeneity as indicated by less than 10%, preferably less than 5%, and more preferably less than 2%, of the total peak area in the experimentally measured PXRD pattern arising from the extra peaks that are absent from the simulated PXRD pattern. Most preferred is a crystalline form having substantially pure phase homogeneity with less than 1% of the total peak area in the experimentally measured PXRD pattern arising from the extra peaks that are absent from the simulated PXRD pattern.

In another embodiment, a composition is provided consisting essentially of the crystalline N-2 form of Compound I. The composition of this embodiment may comprise at least 90 weight % of the crystalline N-2 form of Compound I, based on the weight of Compound I in the composition.

The presence of reaction impurities and/or processing impurities may be determined by analytical techniques known in the art, such as, for example, chromatography, nuclear magnetic resonance spectroscopy, mass spectrometry or infrared spectroscopy.

Crystalline forms may be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of crystalline forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture. An “antisolvent” is a solvent in which the compound has low solubility. Suitable solvents for preparing crystals include polar and nonpolar solvents. High throughput crystallization techniques may be employed to prepare crystalline forms including polymorphs.

Crystals of drugs, including polymorphs, methods of preparation, and characterization of drug crystals are discussed in Solid-State Chemistry of Drugs, S. R. Byrn, R. R. Pfeiffer, and J. G. Stowell, 2nd Edition, SSCI, West Lafayette, Ind. (1999).

For crystallization techniques that employ solvent, the choice of solvent or solvents is typically dependent upon one or more factors, such as solubility of the compound, crystallization technique, and vapor pressure of the solvent. Combinations of solvents may be employed; for example, the compound may be solubilized into a first solvent to afford a solution, followed by the addition of an antisolvent to decrease the solubility of the compound in the solution and to afford the formation of crystals, particularly types and sizes of crystals of interest.

In making the desired form of Compound I, a multi-step process may be used to form various forms and purities of Compound I with the ultimate goal of obtaining the desired form, for example, the N-2 form of Compound I.

In one method to prepare certain particular crystals of Compound I, the H-1 form of Compound I is suspended and/or stirred in a suitable solvent to afford a slurry, which may be heated to promote dissolution. The term “slurry,” as used herein, means a saturated solution of Compound I, which may also contain an additional amount of other polymorphs of Compound I to afford a heterogeneous mixture of Compound I and a solvent at a given temperature. Suitable solvents in this regard include, for example, polar aprotic solvents and polar protic solvents, and mixtures of two or more of these, as disclosed herein. Particular examples of suitable polar aprotic solvents include, but are not limited to, acetonitrile, tetrahydrofuran (THF), dichloromethane, acetone, dimethylformamide, and dimethylsulfoxide.

Seed crystals may be added to any crystallization mixture to promote crystallization. As will be clear to the skilled artisan, seeding is used as a means of controlling growth of a particular crystalline form or as a means of controlling the particle size distribution of the crystalline product. Accordingly, calculation of the amount and types of seeds needed depends on the size of the seed available and the desired size of an average product particle as described, for example, in “Programmed cooling of batch crystallizers,” J. W. Mullin and J. Nyvlt, Chemical Engineering Science (1971) 26:369-377. In general, seeds of small size (for example, in the range of <30 microns) are needed to effectively control the growth of crystals in the batch. Seeds of small size may be generated by sieving, milling, or micronizing larger crystals, or by micro-crystallization of solutions. Care should be taken that milling or micronizing of crystals does not result in any change in crystallinity from the desired crystal form (i.e., change to amorphous or to another polymorph). This control may be done by monitoring with a suitable technique such as Raman.

At the end of the coupling reaction, one has the monohydrate form (H-1 form).

The mixture may then be concentrated (for example, by using distillation (temperature about 50 degrees C.) under vacuum (about 30 Torr), with cooling to rt (about 20-22 degrees C.).

The cooled mixture may be filtered under vacuum, and the isolated solids may be washed with water. The material is then dried under a nitrogen purge to afford the desired crystalline form KF (Karl Fisher) endpoint corresponding to a 1:1 hydrate (H-1 form). Raman is used to monitor form change. Slurry the H-1 material in n-butanol/cyclohexane (9 parts to 1 part=10 L/kg). As this mixture is slurried, it is put through a TURRAX (wet mill). Heptane is added (10 L/kg). The temperature is held at rt (20-22 degrees C.) and filtered. Monitoring is with Raman.

The isolated solids may be analyzed by a suitable spectroscopic or analytical technique, such as PXRD, or the like, known to those skilled in the art, to assure formation of the preferred crystalline form of the product. The resulting crystalline form is typically produced in an amount of greater than about 70 weight % isolated yield, but preferably greater than 90 weight % based on the weight of Compound I originally employed in the crystallization procedure. The product may be co-milled or passed through a mesh screen (for example, using a mesh size in the range of 12-18 screen) to de-lump the product, if necessary, however, the use of a mesh screen is not preferred.

Alternatively, crystalline forms may be obtained by distillation or solvent addition techniques such as those known to those skilled in the art and/or described to in the references listed herein. Suitable solvents for this purpose include any of those solvents described herein, including protic polar solvents, such as alcohols (for example, methanol, ethanol, and isopropanol), aprotic polar solvents (including those listed above), and also ketones (for example, acetone, methyl ethyl ketone, and methyl isobutyl ketone).

By way of general guidance, the reaction mixture may be filtered to remove any undesired impurities, inorganic salts, and the like, followed by washing with reaction or crystallization solvent. The resulting solution may be concentrated to remove excess solvent or gaseous constituents. If distillation is employed, the ultimate amount of distillate collected may vary, depending on process factors including, for example, vessel size, stirring capability, and the like. Suitable temperatures may be used such as in the range of 18-20 degrees C. By way of general guidance, the reaction solution may be distilled to about 1/10 the original volume before solvent replacement is carried out. Solvent replacement may be done using n-butanol/cyclohexane followed by heptane as described above.

The reaction may be sampled and assayed to determine the extent of the reaction and the wt % product in accordance with standard process techniques known to those skilled in the art. If desired, additional reaction solvent may be added or removed to optimize reaction concentration. Preferably, the final concentration is adjusted to about 50 wt % at which point a slurry typically results.

It may be preferable to add solvents directly to the reaction vessel without distilling the reaction mixture. Preferred solvents for this purpose are those which may ultimately participate in the crystalline lattice, as discussed above in connection with solvent exchange. Although the final concentration may vary depending on desired purity, recovery and the like, the final concentration of Compound I in solution is preferably about 4% to about 7%. The reaction mixture may be stirred following solvent addition and simultaneously warmed. By way of illustration, the reaction mixture may be stirred for about 1 hour while warming to about 70° C. The reaction is preferably filtered hot and washed with either the reaction solvent, the solvent added or a combination thereof Seed crystals may be added to any crystallization solution to initiate crystallization.

The various forms described herein may be distinguishable from one another through the use of various analytical techniques known to one of ordinary skill in the art. Such techniques include, but are not limited to, X-ray powder diffraction (PXRD) and/or thermogravimetric analysis (TGA). Specifically, the forms may be characterized and distinguished using single crystal x-ray diffraction, which is based on unit cell measurements of a single crystal of a given form at a fixed analytical temperature. A detailed description of unit cells is provided in Stout & Jensen, X-Ray Structure Determination: A Practical Guide, Macmillan Co., New York (1968), Chapter 3, which is herein incorporated by reference as to such techniques.

Alternatively, the unique arrangement of atoms in spatial relation within the crystalline lattice may be characterized according to the observed fractional atomic coordinates. Another means of characterizing the crystalline structure is by powder x-ray diffraction analysis in which the diffraction profile is compared to a simulated profile representing pure powder material, both run at the same analytical temperature, and measurements for the subject form characterized as a series of 2θ values (usually four or more).

Other means of characterizing the form may be used, such as solid state nuclear magnetic resonance (SSNMR) spectroscopy, differential scanning calorimetry (DSC), thermography and gross examination of the crystalline or amorphous morphology. These parameters may also be used in combination to characterize the subject form.

One of ordinary skill in the art will appreciate that an X-ray diffraction pattern may be obtained with a measurement error that is dependent upon the measurement conditions employed. In particular, it is generally known that intensities in an X-ray diffraction pattern may fluctuate depending upon measurement conditions employed and the shape or morphology of the crystal. It should be further understood that relative intensities may also vary depending upon experimental conditions and, accordingly, the exact order of intensity should not be taken into account. Additionally, a measurement error of diffraction angle for a conventional X-ray diffraction pattern is typically about 0.2% or less, preferably about 0.1% (as discussed hereinafter), and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X-ray diffraction patterns substantially identical to those disclosed in the accompanying Figures fall within the scope of the present invention. In this context, “substantially identical” means that the error of a measurement of diffraction angle for a conventional X-ray diffraction pattern is typically about ±0.2° or less, preferably about ±0.1° or less. Peaks in the powder pattern will be observed to be horizontally in this range, although vertical heights may be different. The ability to ascertain substantial identities of X-ray diffraction patterns is within the purview of one of ordinary skill in the art.

### Utility

The novel crystalline forms of the present invention are selective inhibitors of p38 kinase activity, and in particular, isoforms p38α and p38β. Accordingly, the novel crystalline forms of the invention have utility in treating conditions associated with p38 kinase activity. Such conditions include diseases in which cytokine levels are modulated as a consequence of intracellular signaling via p38, and in particular, diseases that are associated with an overproduction of cytokines IL-1, IL-4, IL-8, and TNF-α. As used herein, the terms “treating” or “treatment” encompass either or both responsive and prophylaxis measures, e.g., measures designed to inhibit or delay the onset of the disease or disorder, achieve a full or partial reduction of the symptoms or disease state, and/or to alleviate, ameliorate, lessen or cure the disease or disorder and/or its symptoms. When reference is made herein to inhibition of “p-38α/β kinase,” this means that either p38α and/or p38β kinase are inhibited. Thus, reference to an IC50 value for inhibiting p-38α/β kinase means that the compound has such effectiveness for inhibiting at least one of, or both of, p38α and p38β kinases.

In view of their activity as inhibitors of p-38α/β kinase, the novel crystalline forms of the invention are useful in treating p-38 associated conditions including, but not limited to, inflammatory diseases, autoimmune diseases, destructive bone disorders, proliferative disorders, angiogenic disorders, infectious diseases, neurodegenerative diseases and viral diseases.

More particularly, the specific conditions or diseases that may be treated with the novel crystalline forms of the invention include, without limitation, pancreatitis (acute or chronic), asthma, allergies, adult respiratory distress syndrome, chronic obstructive pulmonary disease, glomerulonephritis, rheumatoid arthritis, systemic lupus erythematosis, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic β-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoisosis, bone resorption disease, allograft rejections, fever and myalgias due to infection, cachexia secondary to infection, myeloid formation, scar tissue formation, ulcerative colitis, pyresis, influenza, osteoporosis, osteoarthritis and multiple myeloma-related bone disorder, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, cerebral ischemias or neurodegenerative disease caused by traumatic injury; angiogenic disorders including solid tumors, ocular neovasculization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hyposia, vascular hyperplasia, cardiac and renal reperfusion injury, thrombosis, cardiac hypertrophy, thrombin-induced platelet aggregation, endotoxemia and/or toxic shock syndrome, and conditions associated with prostaglandin endoperoxidase syndase-2.

In addition, the novel crystalline p38 inhibitors of this invention inhibit the expression of inducible pro-inflammatory proteins such as prostaglandin endoperoxide synthase-2 (PGHS-2), also referred to as cyclooxygenase-2 (COX-2). Accordingly, additional p38-associated conditions include edema, analgesia, fever and pain, such as neuromuscular pain, headache, pain caused by cancer, dental pain and arthritis pain. The inventive crystalline form also may be used to treat veterinary viral infections, such as lentivirus infections, including, but not limited to, equine infectious anemia virus; or retro virus infections, including feline immunodeficiency virus, bovine immunodeficiency virus and canine immunodeficiency virus.

When the terms “p38 associated condition” or “p38 associated disease or disorder” are used herein, each is intended to encompass all of the conditions identified above as if repeated at length, as well as any other condition that is affected by p38 kinase activity.

The present invention thus provides methods for treating such conditions, comprising administering to a subject in need thereof an effective amount of at least one novel crystalline form of the invention. The methods of treating p38 kinase-associated conditions may comprise administering novel crystalline forms of the invention alone or in combination with each other and/or other suitable therapeutic agents useful in treating such conditions. Exemplary of such other therapeutic agents include corticosteroids, rolipram, calphostin, CSAIDs, 4-substituted imidazo [1,2-A]quinoxalines as disclosed in U.S. Pat. No. 4,200,750; Interleukin-10, glucocorticoids, salicylates, nitric oxide, and other immunosuppressants; nuclear translocation inhibitors, such as deoxyspergualin (DSG); non-steroidal anti-inflammatory drugs (NSAIDs) such as ibuprofen, celecoxib and rofecoxib; steroids such as prednisone or dexamethasone; antiviral agents such as abacavir; antiproliferative agents such as methotrexate, leflunomide, FK506 (tacrolimus, Prograf); cytotoxic drugs such as azathiprine and cyclophosphamide; TNF-α inhibitors such as tenidap, anti-TNF antibodies or soluble TNF receptor, and rapamycin (sirolimus or Rapamune) or derivatives thereof.

The above other therapeutic agents, when employed in combination with the novel crystalline forms of the present invention, may be used, for example, in those amounts indicated in the Physicians' Desk Reference (PDR) or as otherwise determined by one of ordinary skill in the art. In the methods of the present invention, such other therapeutic agent(s) may be administered prior to, simultaneously with, or following the administration of the inventive compounds.

The present invention also provides pharmaceutical compositions containing novel crystalline forms of the invention capable of treating p38-kinase associated conditions, including TNF-α, IL-1, and/or IL-8 mediated conditions, as described above. The inventive compositions may optionally contain other therapeutic agents as described above, and may be formulated, for example, by employing conventional solid or liquid vehicles or diluents, as well as pharmaceutical additives of a type appropriate to the mode of desired administration (e.g., excipients, binders, preservatives, stabilizers, flavors, etc.) according to techniques such as those well known in the art of pharmaceutical formulation.

The novel crystalline forms of the invention may be administered by any means suitable for the condition to be treated, which may depend on the need for site-specific treatment or quantity of drug to be delivered. Topical administration is generally preferred for skin-related diseases, and systematic treatment preferred for cancerous or pre-cancerous conditions, although other modes of delivery are contemplated. For example, the compounds may be delivered orally, such as in the form of tablets, capsules, granules, powders, or liquid formulations including syrups; topically, such as in the form of solutions, suspensions, gels or ointments; sublingually; bucally; parenterally, such as by subcutaneous, intravenous, intramuscular or intrasternal injection or infusion techniques (e.g., as sterile injectable aq. or non-aq. solutions or suspensions); nasally such as by inhalation spray; topically, such as in the form of a cream or ointment; rectally such as in the form of suppositories; or liposomally. Dosage unit formulations containing non-toxic, pharmaceutically acceptable vehicles or diluents may be administered. The compounds may be administered in a form suitable for immediate release or extended release. Immediate release or extended release may be achieved with suitable pharmaceutical compositions or, particularly in the case of extended release, with devices such as subcutaneous implants or osmotic pumps.

Tablets/capsules are preferred.

Exemplary compositions for topical administration include a topical carrier such as PLASTIBASE® (mineral oil gelled with polyethylene).

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art. The inventive compounds may also be orally delivered by sublingual and/or buccal administration, e.g., with molded, compressed, or freeze-dried tablets. Exemplary compositions may include fast-dissolving diluents such as mannitol, lactose, sucrose, and/or cyclodextrins. Also included in such formulations may be high molecular weight excipients such as celluloses (AVICEL®) or polyethylene glycols (PEG); an excipient to aid mucosal adhesion such as hydroxypropyl cellulose (HPC), hydroxypropyl methyl cellulose (HPMC), sodium carboxymethyl cellulose (SCMC), and/or maleic anhydride copolymer (e.g., GANTREZ®); and agents to control release such as polyacrylic copolymer (e.g., CARBOPOL 934®). Lubricants, glidants, flavors, coloring agents and stabilizers may also be added for ease of fabrication and use.

Exemplary compositions for nasal aerosol or inhalation administration include solutions which may contain, for example, benzyl alcohol or other suitable preservatives, absorption promoters to enhance absorption and/or bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, suitable non-irritating excipients, such as cocoa butter, synthetic glyceride esters or polyethylene glycols, which are solid at ordinary temperatures but liquefy and/or dissolve in the rectal cavity to release the drug.

The effective amount of a novel crystalline form of the present invention may be determined by one of ordinary skill in the art, and includes exemplary dosage amounts for a mammal of from about 0.05 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats, horses, and the like. Thus, when the term “patient” is used herein, this term is intended to include all subjects, most preferably mammalian species, that are affected by mediation of p38 enzyme levels.

The novel crystalline forms of the invention, including the compounds described in the examples hereof, may be tested in one or more of the assays described below and may show activity as inhibitors of p38α/β enzymes and TNF-α.

### Biological Assays

### Generation of p38 Kinases

cDNAs of human p38α, β and γ isozymes are cloned by PCR. These cDNAs are subcloned in the pGEX expression vector (Pharmacia). GST-p38 fusion protein is expressed in E. Coli and purified from bacterial pellets by affinity chromatography using glutathione agarose. p38 fusion protein is activated by incubating with constitutively active MKK6. Active p38 is separated from MKK6 by affinity chromatography. Constitutively active MKK6 is generated according to Raingeaud, et al. [Mol. Cell. Biol., 1247-1255 (1996)].

### TNF-α Production by LPS-Stimulated PBMCs

Heparinized human whole blood is obtained from healthy volunteers. Peripheral blood mononuclear cells (PBMCs) are purified from human whole blood by Ficoll-Hypaque density gradient centrifugation and resuspended at a concentration of 5×10⁶/ml in assay medium (RPMI medium containing 10% fetal bovine serum). 50 μL of cell suspension is incubated with 50 μL of test compound (4× concentration in assay medium containing 0.2% DMSO) in 96-well tissue culture plates for 5 minutes at RT. 100 μL of LPS (200 ng/ml stock) is then added to the cell suspension and the plate is incubated for 6 hours at 37° C. Following incubation, the culture medium is collected and stored at −20° C. TNF-α concentration in the medium is quantified using a standard ELISA kit (Pharmingen-San Diego, Calif.). Concentrations of TNF-α and IC₅₀ values for test compounds (concentration of compound that inhibited LPS-stimulated TNF-α production by 50%) are calculated by linear regression analysis.

### p38 Assay

The assays are performed in V-bottomed 96-well plates. The final assay volume is 60 μL prepared from three 20 μL additions of enzyme, substrates (MBP and ATP) and test compounds in assay buffer (50 mM Tris pH 7.5, 10 mM MgCl₂, 50 mM NaCl and 1 mM DTT). Bacterially expressed, activated p38 is pre-incubated with test compounds for 10 min. prior to initiation of reaction with substrates. The reaction is incubated at 25° C. for 45 min. and terminated by adding 5 μL of 0.5 M EDTA to each sample. The reaction mixture is aspirated onto a pre-wet filtermat using a Skatron Micro96 Cell Harvester (Skatron, Inc.), then washed with PBS. The filtermat is then dried in a microwave oven for 1 min., treated with MeltilLex A scintillation wax (Wallac), and counted on a Microbeta scintillation counter Model 1450 (Wallac). Inhibition data are analyzed by nonlinear least-squares regression using Prizm (GraphPadSoftware). The final concentration of reagents in the assays are ATP, 1 μM; [γ-³³P]ATP, 3 nM; MBP (Sigma, #M1891), 2 μg/well; p38, 10 nM; and DMSO, 0.3%.

### TNF-α Production by LPS-Stimulated Mice

Mice (Balb/c female, 6-8 weeks of age, Harlan Labs; n=8/treatment group) are injected intraperitoneally with 50 μg/kg lipopolysaccharide (LPS; E coli strain 0111:B4, Sigma) suspended in sterile saline. Ninety minutes later, mice are sedated by CO₂:O₂ inhalation and a blood sample is obtained. Serum is separated and analyzed for TNF-alpha concentrations by commercial ELISA assay per the manufacturer's instructions (R&D Systems, Minneapolis, Minn.).

Test compounds are administered orally at various times before LPS injection. The compounds are dosed either as suspensions or as solutions in various vehicles or solubilizing agents.

### Abbreviations

For ease of reference, the following abbreviations are employed herein, including the methods of preparation and Examples that follow:
μL=microliter
μL or μl=microliter
μM=micromolar
API=active pharmaceutical ingredient
aq.=aqueous
Boc=tert-butyloxycarbonyl
BuOAc=butyl acetate
Bz=benzyl
DCE=1,2-dichloroethane
DCM=dichloromethane
DIPEA=diisopropylethylamine
DMA=N,N-dimethyl acetamide
DMF=N,N-dimethyl formamide
DMSO=dimethyl sulfoxide
DTT=dithiothreitol
EDC or EDCI=1-(3-Dimethylaminopropyl)-3-ethylcarbodiimide hydrochloride
Et=ethyl
EtOAc=ethyl acetate
EtOH=ethanol
g=gram(s)
HATU=O-(7-Azabenzotriazol-1-yl-N,N,N′,N′-tetramethyluronim hexafluorophosphate
HOBt=1-hydroxybenzotriazole hydrate
HPLC=high performance liquid chromatography
iPrOAc=isopropyl acetate
Iso-P=isopropyl
K₂CO₃=potassium carbonate
KOH=potassium hydroxide
L=liter
LC/MS=high performance liquid chromatography/mass spectrometry
LOD=loss on dry or loss on drying
m-CPBA=m-chloroperbenzoic acid
Me=methyl
MeOH=methanol
meq=milliequivalent
mg=milligram(s)
min=minute(s)
ml or mL=milliliter
mM=millimolar
mmol=millimole(s)
mol=moles
mp=melting point
MS=mass spectrometry
NaH=sodium hydride
NaOH=sodium hydroxide
ng=nanogram
NIST=National Institute for Standards and Technology
nM=nanomolar
NMR=nuclear magnetic resonance
Pd=palladium
Pd/C=palladium on carbon
Ph=phenyl
POCl₃=phosphorous oxychloride
Pr=propyl
RAP=relative area percent
ret. t.=HPLC retention time (minutes)
RH=relative humidity
RP HPLC=reverse phase HPLC
RT or rt=room temperature (20 to 25° C.)
sat or sat'd=saturated
t-Bu=tertiary butyl
TFA=trifluoroacetic acid
THF=tetrahydrofuran
TLC=thin layer chromatography

In the Examples, designations associated with HPLC data reflect the following conditions:

Method A. Column: YMC ODSA S-5 5 u C18 4.6×50 mm; Solvent: solvent A=10% MeOH/90% water/0.1% THF, and solvent B=90% MeOH/10%water/0.1% THF; Method: 4 min gradient;

Method B. Column: YMC s5 ODS 4.6×50 mm; Solvent: solvent A=10% MeOH/90% water/0.2% H₃PO₄, and solvent B=90% MeOH/10% water/0.2% H₃PO₄; Method: 4 min gradient.

### Preparation of N-2 Crystalline Form of Compound I

While one method of obtaining the N-2 form of Compound I by crystallization from BuOAc, iPrOAc and acetone is described above, a preferred method of obtaining the N-2 form is described in Scheme 1 below.

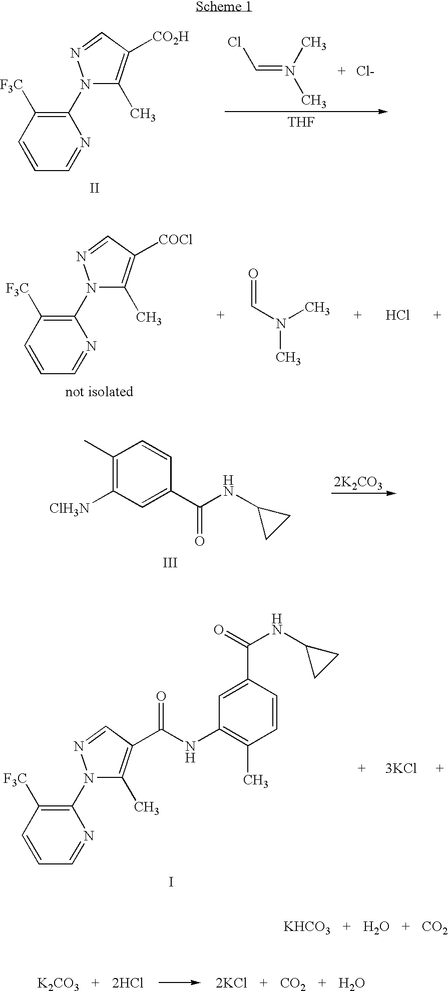

### Scheme 2

Repeat Scheme 1 but substitute sodium phosphate dibasic for the potassium carbonate.

### Procedure for Characterizing the Forms

### Single Crystal Data

Data were collected on a Bruker-Nonius¹ CAD4 serial diffractometer. Unit cell parameters were obtained through least-squares analysis of the experimental diffractometer settings of 25 high-angle reflections. Intensities were measured using Cu Kα radiation (λ=1.5418 Å) at a constant temperature with the Θ-2Θ variable scan technique and were corrected only for Lorentz-polarization factors. Background counts were collected at the extremes of the scan for half of the time of the scan. Alternately, single crystal data were collected on a Bruker-Nonius Kappa CCD 2000 system using Cu Kα radiation (λ=1.5418 Å). Indexing and processing of the measured intensity data were carried out with the HKL2000 software package² in the Collect program suite.³ ¹BRUKER AXS, Inc., 5465 East Cheryl Parkway, Madison, Wis. 53711 USA.²Otwinowski, Z. & Minor, W. (1997) in Macromolecular Crystallography, eds. Carter, W. C. Jr & Sweet, R. M. (Academic, NY), Vol. 276, pp. 307-326.³Collect Data collection and processing user interface: Collect: Data collection software, R. Hooft, Nonius B. V., 1998.

When indicated, crystals were cooled in the cold stream of an Oxford cryo system⁴ during data collection. ⁴Oxford Cryosystems Cryostream cooler: J. Cosier and A. M. Glazer, J. Appl. Cryst., 1986, 19, 105.

The structures were solved by direct methods and refined on the basis of observed reflections using either the SDP⁵ software package with minor local modifications or the crystallographic package, MAXUS.⁶ ⁵SDP, Structure Determination Package, Enraf-Nonius, Bohemia N.Y. 11716 Scattering factors, including f′ and f″, in the SDP software were taken from the “International Tables for Crystallography”, Kynoch Press, Birmingham, England, 1974, Vol IV, Tables 2.2A and 2.3.1.⁶maXus solution and refinement software suite: S. Mackay, C. J. Gilmore, C. Edwards, M. Tremayne, N. Stewart, K. Shankland. maXus: a computer program for the solution and refinement of crystal structures from diffraction data.

The derived atomic parameters (coordinates and temperature factors) were refined through full matrix least-squares. The function minimized in the refinements was Σw(|Fo|−|Fc|)². R is defined as Σ∥Fo|−|Fc∥/Σ |Fo|, while Rw=[Σw(|Fo|−|Fc|)²/Σw |Fo|²]^{1/2}, where w is an appropriate weighting function based on errors in the observed intensities. Difference maps were examined at all stages of refinement. Hydrogens were introduced in idealized positions with isotropic temperature factors, but no hydrfogen parameters were varied.

### PXRD

X-ray powder diffraction (PXRD) data were obtained using a Bruker C2 GADDS. The radiation was Cu Kα (40 KV, 50 mA). The sample-detector distance was 15 cm. Powder samples were placed in sealed glass capillaries of 1 mm or less in diameter; the capillary was rotated during data collection. Data were collected for 3≦2θ≦35° with a sample exposure time of at least 2000 seconds. The resulting two-dimensional diffraction arcs were integrated to create a traditional 1-dimensional PXRD pattern with a step size of 0.02 degrees 2θ in the range of 3 to 35 degrees 2θ.

### DSC

Differential scanning calorimetry (DSC) experiments were performed in a TA Instruments™ model Q1000 or 2920. The sample (about 2-6 mg) was weighed in an aluminum pan and recorded accurately recorded to a hundredth of a milligram, and transferred to the DSC. The instrument was purged with nitrogen gas at 50 mL/min. Data were collected between room temperature and 300° C. at 10° C./min heating rate. The plot was made with the endothermic peaks pointing down.

### TGA

Thermal gravimetric analysis (TGA) experiments were performed in a TA Instruments™ model Q500 or 2950. The sample (about 10-30 mg) was placed in a platinum pan previously tared. The weight of the sample was measured accurately and recorded to a thousandth of a milligram by the instrument. The furnace was purged with nitrogen gas at 100 mL/min. Data were collected between room temperature and 300° C. at 10° C./min heating rate.

### EXAMPLES

The following Examples are offered as illustrative as a partial scope of the invention, including preferred embodiments, but are not meant to be limiting of the scope of the invention. Unless otherwise indicated, they have been prepared, isolated and characterized using the methods disclosed herein. The abbreviations used herein are defined above. The analytical methods used are as described herein.

### Example A—Compound I

### Step 1: Coupling Step to Prepare Crude Compound I and Removing Some Impurities

In a reactor was charged 1 kg of Compound II, 0.5 kg of Vilsmeier reagent and 7.1 kg (8 mL/g Compound II input) of THF. The resulting slurry was agitated for 1-2 h at 20° C. Complete dissolution was observed after about 0.5 h. HPLC analysis indicated that less than 0.5 relative area percent (RAP) of Compound II remained (IPC specification RAP 4.0%; see analytical section for HPLC method).

In a separate glass reactor was charged 0.9 kg of Compound III, 7.4 kg of 1.35 M potassium phosphate dibasic, 4.4 kg of (8 mL/g Compound II input) THF. The resulting slurry was agitated for 1 h at 20° C. after which time all of the solids had completely dissolved and a homogeneous biphasic mixture was obtained. The batch temperature was set to 20° C. and the rich Compound II-acid chloride stream was added to the biphasic mixture maintaining the batch temperature at less than 25° C. On lab scale, the addition took 0.5 h. (Note: The acid chloride stream has been added over a 1 min. period with a concomitant exotherm to 28° C. without negatively affecting yield or quality.) The resulting solution was agitated for 1 h after which time HPLC analysis indicated that less than 0.5 RAP of the acid chloride remained (IPC specification RAP<1.0%; see analytical section for HPLC method). The apparent pH was adjusted to 6.8 (range 6.2-7.2) with 2.1 kg of 5N sodium hydroxide. The batch was heated to 35° C. Agitation was stopped and the layers were allowed to separate. The lower spent aqueous stream was removed. The batch temperature was set to 40° C. The rich THF stream was concentrated under vacuum to about half of the original volume while maintaining the batch temperature between 35-50° C. To the resulting product slurry was added 5 kg (12 mL/g Compound II input) of water while maintaining the temperature between 30-40° C. The slurry was cooled to 20° C. and held at this temperature for at least 1 h. The crystals were collected via filtration and washed with water to a conductivity endpoint of less than 100μ Siemens/cm. The cake was deliquored and dried under vacuum at 50° C. to an LOD endpoint of less than 4.0 wt %. The crude was isolated in 97-99% yield with 99.8-100.00 RAP purity.

### Step 2: Polymorph Transformation

The second step in the process is a polymorph transformation with concomitant control of particle size. Two processes were developed for the polymorph transformation and to control the particle size. These are designated herein as Process A and Process B.

In Process A, the Compound I that is first isolated (referred to as “crude” or “first drop”) directly from the Schotten-Baumen coupling was dried to an endpoint that corresponds to about a 1:1 hydrate (the H-1 form). This was then completely dissolved in hot (about 80 degrees C.) 1-butanol (about 9 mL/g crude Compound I) and seeded with Compound I seed crystals which had been previously milled to a particle size D[90]<20 μm. Process A achieves the desired N-2 form but further work is needed to achieve a better particle size. Process A was used in a batch to give the N-2 form of Compound I having a primary particle size D[90]>65 μm, which was well outside of the desired particle size specification of particle size. Heptane is then added to maximize the yield. This has no effect on the polymorph or primary particle size within the established target specifications. Particular goals for particle sizes being D[90]<30 μm and more particularly D[90]<20 μm.

Process B is used to improve the particle size. In Process B, the first drop Compound I can be over-dried at a temp such as above 50-60 degrees C. in a dryer (many types of dryers being available) to give a mixture of 2 metastable forms, N-7 and H-1. This mixture is slurried in a solvent composition of 1-butanol and cyclohexane 9:1 at ambient temperature as it is circulated through a wet mill. Specifically, 1.0 kg of Compound I crude, 4.9 kg (6 L/kg input) of 1-butanol and 0.8 kg (1 L/kg input) of cyclohexane was charged to a crystallizer. The slurry was agitated and the batch temperature was set to 20-25 degrees C. The reactor had a pump around loop with a wet mill, Raman probe and Lasentec FBRM probe. The wet mill was turned-on and the batch was re-circulated through the wet mill and pump around loop. The Raman probe in the pump around loop was used to monitor the form transformation. (Note: In the laboratory, the polymorph transformation was typically complete within 2 h.) After form conversion was verified by XRD, 2.1 kg (3 L/kg input) of n-heptane was added over a 1 h period and the slurry was agitated for an additional 1 h. This has no effect on the polymorph or primary particle size within the established specifications of D[90]<30 μm. The product was collected via filtration and washed with 1.7 kg (2.5 L/kg) of n-heptane. The cake was deliquored and dried under vacuum (about 10 Torrr) at 50-55° C. to an endpoint of <0.5 wt % 1-butanol and n-heptane by GC. The API was isolated in 84-91% yield with 99.8-100.00 RAP purity. This produces the desired neat N-2 form having a primary particle size D[90]<20 μm.

### Example B Form Change to N-2

For a form change of Compound I to N-2, the following procedure may be used.
1) Set up an in-line Raman Spectrometer for a 4 L crystallizer fitted with a recirculation loop containing a TURRAX (wet mill) (model UTL25) from the bottom valve of the crystallizer to a port at the top of the crystallizer.
2) Charge 12 g of Compound I hydrate to he crystallizer.
3) Charge 1.125 L (10 L/kg input) of 1-butanol to the crystallizer.
4) Charge 1.125 L (1 L/kg input) of cyclohexane to the crystallizer and begin agitation.
5) Agitate the slurry at 18-20 degrees C. with TURRAX (wet mill) (contains a fine dispersion element) set at 1.
6) Monitor the form change by Raman analysis.
7) Sample the slurry to monitor particle size by visual microscopic analysis (may be completed within 2 h.).
8) Charge 1.5 L (1.2 L/kg input) of heptane to he slurry over a 1 h period.
9) Agitate the slurry at 18-20 degrees C. for 1 h.
10) Filter the batch (typical concentration will be 2-2.5 mg/mL).
11) Wash with about 2 cake volumes of heptane.
12) Deliquor the cake for at least 1 h.
13) Dry the cake at 50-55 degrees overnight to ensure butanol concentration is <0.5% by GC analysis.

### Examples 1-13

The crystalline form of Compound I was prepared and is tabulated as Examples 1-6 shown in Table 1 below. Other forms of Compound I are seen in Examples 7-13. Said crystalline form comprises crystals of form N-2 (neat form). Each Example listed in Table 1 show a different way to analyze Form N-2 of Compound I using one or more of the testing methods described hereinabove.

TABLE 1

| Example | Form | Solvents | Type |
|---|---|---|---|
| 1 | N-2 | n-BuOAc @80° C., acetone and | Neat crystal |
| | | iPrOAc/heptane @rt | |
| 2 | H-1 | aq. MeOH (MFM) | Monohydrate |
| 3 | N-7 | (heat 90° C., 30 m) topotactic from | Neat crystal |
| | (T1H1) | H-1 | |
| 4 | N-5 | (from the melt) Heat H-1 to | Neat crystal |
| | | ~215° C. | |
| 5 | N-6 | (from the melt) Heat Lot 3 to | Neat crystal |
| | | ~220° C. | |
| 6 | P-14 | Aq. THF | |
| 7 | AN-3 | MeCN | MeCN solvate |
| 8 | E-8 | EtOH | ethanolate |
| 9 | EA.5-3 | EtOAc/heptane | EtOAc |
| | | | solvate |
| 10 | IPA-10 | IPA | isopropanol |
| | | | solvate |
| 11 | SA-9 | Aq. THF | Solvate |
| 12 | SC-13 | Aq. THF | Solvate |
| 13 | SD-14 | Aq. THF | Solvate |

### Example 1

### A. Single Crystal X-Ray Measurements

Following the above Single Crystal Data procedure, the approximate unit cell dimensions in Angstroms (Å), as measured at a sample temperature of 22° C., as well as the crystalline cell volume (V), space group (sg), molecules per unit cell, and crystal density for the N-2 form of Compound I are shown below.

Cell dimensions: a =17.976(2) Å
b =12.530(1) Å
c=19.639(2) Å
α=90°
β=105.03(1)°
γ=90°
Volume=4272(1) Å³

Space group: C2/c

Molecules/unit cell (Z): 8

Density, calc g-cm⁻³: 1.379

The unit cell data and other properties for these examples are tabulated above. The unit cell parameters were obtained from single crystal X-ray crystallographic analysis according to the procedure described in Stout & Jensen, “X-Ray Structure Determination: A Practical Guide”, (MacMillian, 1968), previously herein incorporated by reference. The fractional atomic coordinates for the N-2 form of Compound I are tabulated in Table 2 hereinbelow. The derived atomic parameters (coordinates and temperature factors) for all examples herein were refined through full matrix least-squares. The function minimized in the refinements was Σ_{w}(|Fₒ|−|F_{c}|)². R is defined as Σ ∥F|−|F∥/Σ |Fₒ|, while R_{w}=[Σ_{w}(|Fₒ|−|F_{c})²/Σ_{w} |Fₒ|²]^{1/2}, where w is an appropriate weighting function based on errors in the observed intensities. Difference maps were examined at all stages of refinement. Hydrogen atoms were introduced in idealized positions with isotropic temperature factors, but no hydrogen parameters were varied.

A moisture sorption study indicates that the Form N-2 is non-hygroscopic in the range from about 25 to about 75% RH at 25° C.

### B. Fractional Atomic Coordinates

The arrangement of the Compound I molecules in the N-2 form may additionally be characterized by the approximate fractional atomic coordinates listed in Table 2. The approximate coordinates in Table 2 will therefore vary according to the temperature at measurement. Statistical variations in these coordinates may also occur consistent with the reported error values.

TABLE 2
Fractional Atomic Parameters and Their Estimated Standard
Deviations for Form N-2 of Compound I at rt

| Atom | x | y | z | B(iso) |
|---|---|---|---|---|
| F28 | 0.00631(17) | 0.3124(2) | −0.13523(13) | 8.3 |
| F29 | −0.0320(2) | 0.1722(2) | −0.09611(14) | 9.7 |
| F30 | −0.11286(15) | 0.2848(3) | −0.15238(12) | 9.0 |
| O13 | 0.17211(14) | −0.00444(17) | 0.18223(10) | 4.6 |
| O22 | 0.28400(15) | −0.43821(19) | 0.05224(10) | 5.1 |
| N1 | 0.05957(15) | 0.22704(19) | 0.03594(13) | 4.0 |
| N2 | 0.11923(17) | 0.2418(2) | 0.00505(14) | 4.7 |
| N8 | −0.00098(17) | 0.3548(2) | 0.08629(14) | 4.9 |
| N14I | 0.23277(15) | −0.04264(19) | 0.09708(12) | 3.9 |
| N23I | 0.3016(2) | −0.5015(2) | 0.16207(13) | 6.1 |
| C3I | 0.1667(2) | 0.1630(3) | 0.03066(16) | 4.5 |
| C4 | 0.13996(19) | 0.0986(2) | 0.07791(14) | 3.8 |
| C5 | 0.07025(18) | 0.1414(2) | 0.08029(14) | 3.8 |
| C6I | 0.0111(2) | 0.1067(3) | 0.11604(19) | 5.1 |
| C7 | 0.00167(18) | 0.3069(2) | 0.02721(16) | 4.0 |
| C9I | −0.0515(2) | 0.4344(3) | 0.0814(2) | 5.5 |
| C10I | −0.0987(2) | 0.4692(3) | 0.0202(2) | 5.8 |
| C11I | −0.0968(2) | 0.4166(3) | −0.0409(2) | 5.4 |
| C12 | −0.04623(19) | 0.3322(3) | −0.03839(17) | 4.4 |
| C13 | 0.18158(18) | 0.0126(2) | 0.12337(14) | 3.7 |
| C15 | 0.27968(17) | −0.1254(2) | 0.13589(13) | 3.4 |
| C16 | 0.33525(19) | −0.1026(2) | 0.19938(15) | 4.2 |
| C17I | 0.3769(2) | −0.1883(3) | 0.23503(17) | 4.8 |
| C18I | 0.3657(2) | −0.2909(3) | 0.21021(16) | 4.7 |
| C19 | 0.31351(18) | −0.3123(2) | 0.14590(14) | 3.8 |
| C20I | 0.27151(17) | −0.2279(2) | 0.10872(14) | 3.6 |
| C21I | 0.3501(2) | 0.0090(3) | 0.22801(19) | 5.7 |
| C22 | 0.2996(2) | −0.4222(3) | 0.11606(15) | 4.2 |
| C24I | 0.2831(3) | −0.6139(3) | 0.14021(19) | 7.9 |
| C25I | 0.3413(4) | −0.6915(4) | 0.1569(3) | 8.9 |
| C26I | 0.2844(3) | −0.6924(3) | 0.1935(2) | 7.6 |
| C27 | −0.0459(2) | 0.2738(3) | −0.1044(2) | 5.8 |

### C. Powder X-Ray Diffraction

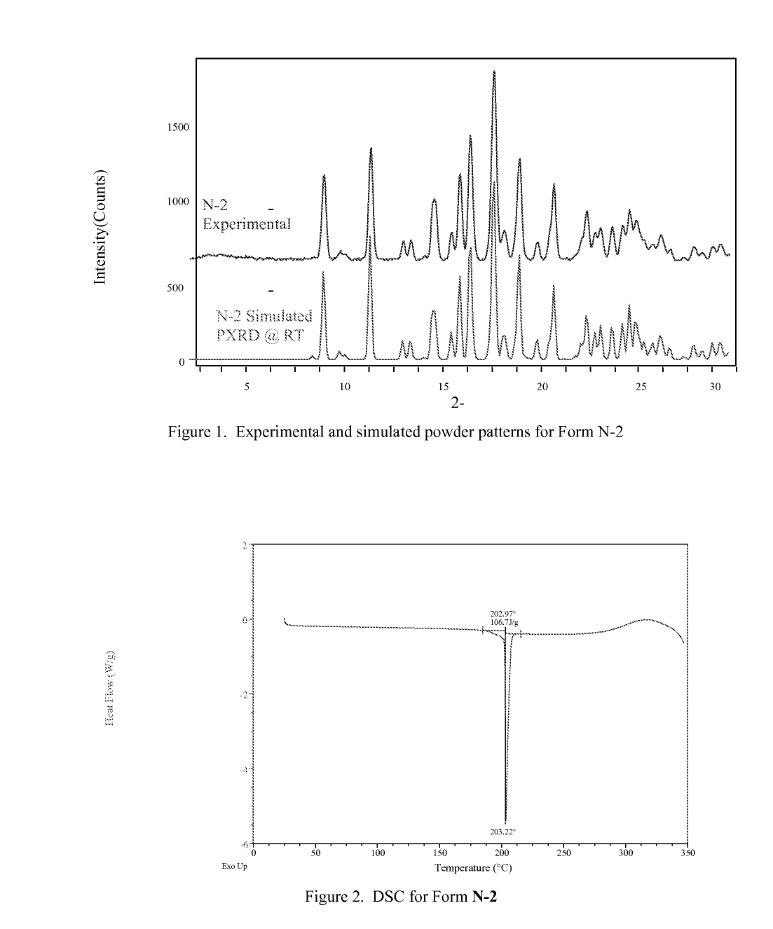

X-ray powder diffraction (PXRD) data were obtained using the PXRD procedure described hereinabove. Table 3 and FIG. 1 show the PXRD data for the N-2 crystalline form for Compound I.

TABLE 3
Characteristic diffraction peak positions (degrees 2θ ± 0.1)@
RT, based on a high quality pattern collected with a diffractometer
(CuKα) with a spinning capillary with 2 theta (“2θ”)
calibrated with a NIST or other suitable standard.

| Peak # | 2-Theta (°) |
|---|---|
| 1 | 9.3 |
| 2 | 11.9 |
| 3 | 15.4 |
| 4 | 16.9 |
| 5 | 17.5 |
| 6 | 18.8 |
| 7 | 20.2 |
| 8 | 22.1 |

### D. Differential Scanning Calorimetry (DSC)

Differential scanning calorimetry was conducted for each crystalline form using a TA Instruments™ model Q1000. For each analysis, the DSC cell/sample chamber was purged with 50 mL/min from above of ultra-high purity nitrogen gas. The instrument was calibrated with high purity indium. The heating rate was 10° C. per minute in the temperature range between 25 and 300° C. The heat flow, which was normalized by sample weight, was plotted versus the measured sample temperature. The data were reported in units of watts/gram (“W/g”). The plot was made with the endothermic peaks pointing down. The endothermic melt peak (melting point) was evaluated for extrapolated onset temperature. FIG. 2 shows the DSC thermogram for the N-2 crystal form of Compound I, which was observed to have an endothermic transition with an onset in the range from about 201° C. to about 205° C.

### E. Thermogravimetric Analysis (TGA)

Thermogravimetric analysis was conducted using the procedure described above. FIG. 3 shows the TGA curve for the N-2 crystal form of Compound I, which has a negligible weight loss up to about 180° C.

### Example 2

### A. Single Crystal X-Ray Measurements

Following the above Single Crystal Data procedure, the approximate unit cell dimensions in Angstroms (Å), as measured at a sample temperature of 22° C., as well as the crystalline cell volume (V), space group (sg), molecules per unit cell, and crystal density for the H-1 form of Compound I are shown below.

Cell dimensions: a=11.023(1) Å
b=12.080(2) Å
c=9.026(1) Å
α=109.88(1)°
β=90.14(1)°
γ=92.79(1)°
Volume=1128.6(5) Å³

Space group: P-1

Molecules/unit cell (Z): 2

Density, calc g-cm⁻³: 1.358

The fractional atomic coordinates for the H-1 form of Compound I are tabulated in Table 4 hereinbelow. A moisture sorption study indicates that the Form H-1 is essentially non-hygroscopic in the range from about 35 to about 75% RH.

### B. Fractional Atomic Coordinates

The arrangement of the Compound I molecules in the H-1 form may additionally be characterized by the approximate fractional atomic coordinates listed in Table 4 below. The approximate coordinates in Table 4 will therefore vary according to the temperature at measurement. Statistical variations in these coordinates may also occur consistent with the reported error values.

TABLE 4
Fractional Atomic Parameters and Their Estimated Standard
Deviations for Form H-1 of Compound I at rt

| Atom | x | Y | z | B(iso) |
|---|---|---|---|---|
| F28 | 0.6224(3) | 0.7227(4) | 0.2190(7) | 11.7(2) |
| F29 | 0.4721(5) | 0.6357(4) | 0.0472(6) | 14.1(1) |
| F30 | 0.5555(4) | 0.7879(3) | 0.0284(4) | 9.4(1) |
| F100 | 0.604(1) | 0.7950(8) | 0.096(1) | 11.9(3) |
| F200 | 0.5898(9) | 0.6837(9) | 0.179(1) | 8.3(3) |
| F300 | 0.4825(7) | 0.6231(5) | 0.0399(8) | 5.2(2) |
| O13 | 0.0901(2) | 0.3340(2) | 0.1392(3) | 4.37(5) |
| O22 | 0.3291(2) | −0.1378(2) | −0.1559(3) | 6.16(7) |
| O99 | 0.4812(2) | 0.2355(2) | 0.3492(3) | 4.40(5) |
| N1 | 0.3312(2) | 0.6375(2) | 0.2922(3) | 3.38(5) |
| N2 | 0.4205(2) | 0.5911(2) | 0.3535(3) | 4.36(6) |
| N12 | 0.2617(3) | 0.8234(2) | 0.4124(4) | 4.71(7) |
| N14 | 0.2415(2) | 0.2548(2) | 0.2332(3) | 3.40(6) |
| N23 | 0.1631(2) | −0.2590(2) | −0.1748(3) | 4.00(6) |
| C3 | 0.3811(3) | 0.4797(2) | 0.3232(4) | 4.32(8) |
| C4 | 0.2674(2) | 0.4549(2) | 0.2456(3) | 3.06(6) |
| C5 | 0.2366(3) | 0.5588(2) | 0.2272(3) | 3.13(6) |
| C6 | 0.1298(3) | 0.5916(3) | 0.1546(4) | 4.88(8) |
| C7 | 0.3418(3) | 0.7608(2) | 0.3156(4) | 3.38(7) |
| C8 | 0.4316(3) | 0.8075(3) | 0.2458(4) | 4.13(8) |
| C9 | 0.4392(3) | 0.9293(3) | 0.2830(4) | 4.86(8) |
| C10 | 0.3592(3) | 0.9957(3) | 0.3852(4) | 4.94(8) |
| C11 | 0.2725(3) | 0.9412(3) | 0.4463(5) | 5.4(1) |
| C13 | 0.1924(3) | 0.3429(2) | 0.2001(4) | 3.24(7) |
| C15 | 0.1816(2) | 0.1390(2) | 0.1928(4) | 3.15(6) |
| C16 | 0.0874(3) | 0.1183(2) | 0.2842(4) | 3.50(7) |
| C17 | 0.0337(3) | 0.0043(3) | 0.2366(4) | 3.94(7) |
| C18 | 0.0750(3) | −0.0861(2) | 0.1082(4) | 3.64(7) |
| C19 | 0.1722(2) | −0.0645(2) | 0.0241(4) | 3.28(7) |
| C20 | 0.2252(3) | 0.0501(2) | 0.0674(4) | 3.32(7) |
| C21 | 0.0469(3) | 0.2119(3) | 0.4312(4) | 4.88(9) |
| C22 | 0.2283(3) | −0.1561(2) | −0.1095(4) | 3.92(8) |
| C24 | 0.2086(3) | −0.3510(3) | −0.3068(4) | 4.60(8) |
| C25 | 0.2685(4) | −0.4500(3) | −0.2803(6) | 7.4(1) |
| C26 | 0.1458(4) | −0.4686(3) | −0.3496(6) | 7.1(1) |
| C27 | 0.5198(4) | 0.7352(4) | 0.1337(6) | 7.1(1) |

### C. Powder X-Ray Diffraction

X-ray powder diffraction (PXRD) data were obtained using the PXRD procedure described hereinabove. Table 5 and FIG. 4 show the PXRD data for the H-1 form of Compound I.

TABLE 5
Characteristic diffraction peak positions (degrees 2θ ± 0.1)@ RT,
based on a high quality pattern collected with a diffractometer
(CuKα) with a spinning capillary with 2θ calibrated with
a NIST other suitable standard.

| Peak # | 2-Theta (°) |
|---|---|
| 1 | 8.0 |
| 2 | 11.5 |
| 3 | 13.3 |
| 4 | 17.3 |
| 5 | 17.9 |
| 6 | 19.2 |
| 7 | 15.5 |
| 8 | 26.7 |

### D. Differential Scanning Calorimetry (DSC)

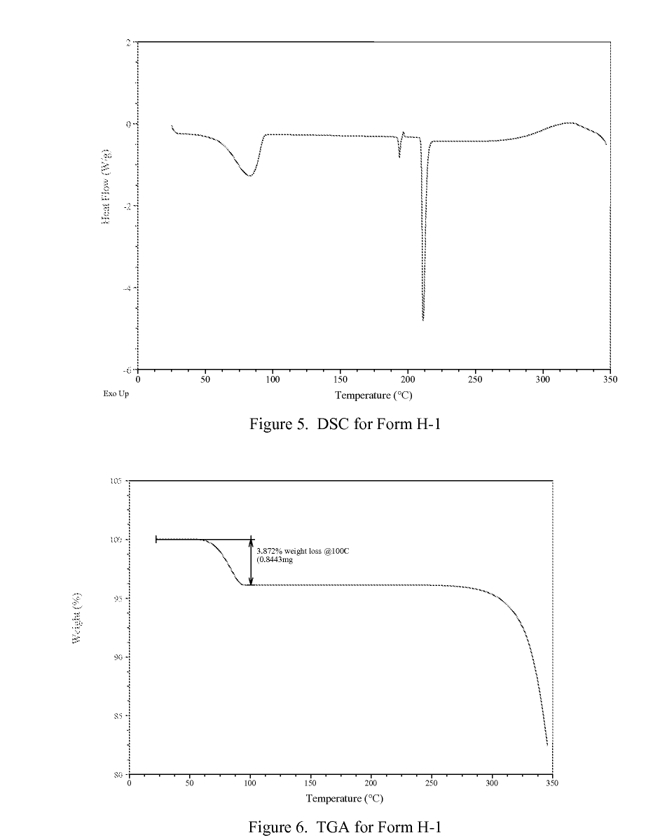

Differential scanning calorimetry was conducted using the procedure described above. FIG. 5 shows the DSC thermogram for the H-1 crystal form of Compound I.

### E. Thermogravimetric Analysis (TGA)

Thermogravimetric analysis was conducted using the procedure described above. FIG. 6 shows the TGA curve for the H-1 crystal form of Compound I, which was observed to have weight loss corresponding to one mole of water per mole of drug.

### Example 3

### A. Single Crystal X-Ray Measurements

Following the above Single Crystal Data procedure, the approximate unit cell dimensions in Angstroms (Å), as measured at a sample temperature of 22° C., as well as the crystalline cell volume (V), space group (sg), molecules per unit cell, and crystal density for the N-7 form of Compound I are shown below.

Cell dimensions: a=9.38(4) Å
b=11.70(1) Å
c=11.85(4) Å
α=123.9(3)°
β=81.1(3)°
γ=93.5(3)°
Volume=1065(17) Å³

Space group: P-1

Molecules/unit cell (Z): 2

Density, calc g-cm⁻³: 1.383

### B. Powder X-Ray Diffraction

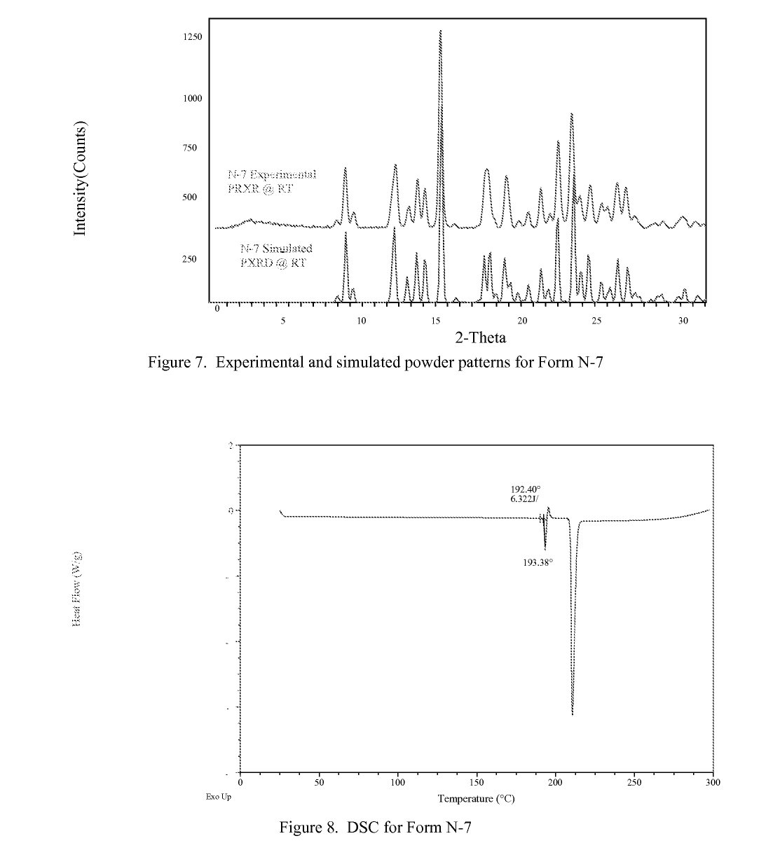

X-ray powder diffraction (PXRD) data were obtained using the PXRD procedure described hereinabove. FIG. 7 and Table 6 show the PXRD data for the N-7 form of Compound I.

TABLE 6
Characteristic diffraction peak positions (degrees 2θ ± 0.1)@ RT,
based on a high quality pattern collected with a diffractometer
(CuKα) with a spinning capillary with 2θ calibrated
with a NIST other suitable standard.

| Peak # | 2-Theta (°) |
|---|---|
| 1 | 9.1 |
| 2 | 12.2 |
| 3 | 13.6 |
| 4 | 14.1 |
| 5 | 15.2 |
| 6 | 18.3 |
| 7 | 22.6 |
| 8 | 23.6 |

### D. Differential Scanning Calorimetry (DSC)

Differential scanning calorimetry was conducted using the procedure described above. FIG. 8 shows the DSC thermogram for the N-7 crystal form of Compound I, which was observed to have an endothermic transition with an onset in the range from about 190° C. to about 194° C.

### D. Thermogravimetric Analysis (TGA)

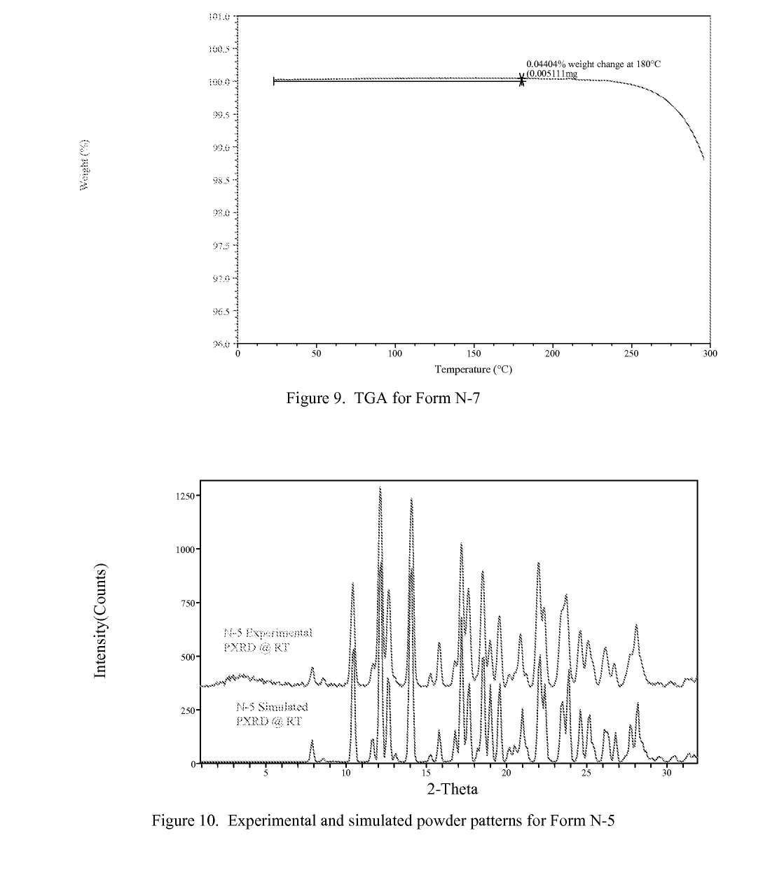

Thermogravimetric analysis was conducted using the procedure described above. FIG. 9 shows the TGA curve for the N-7 crystal form of Compound I, which has a negligible weight loss up to about 180° C.

### Example 4

### A. Single Crystal X-Ray Measurements

Following the above Single Crystal Data procedure, the approximate unit cell dimensions in Angstroms (Å), as measured at a sample temperature of 22° C., as well as the crystalline cell volume (V), space group (sg), molecules per unit cell, and crystal density for the N-5 form of Compound I are shown below.

Cell dimensions: a=26.406(3) Å
b=11.638(3) Å
c=17.204(3) Å
α=90°
β=121.77(1)°
γ=90°
Volume=4495(1) Å³

Space group: C2/c

Molecules/unit cell (Z): 8

Density, calc g-cm⁻³: 1.310

The fractional atomic coordinates for the N-5 form of Compound I are tabulated in Table 7 hereinbelow.

### B. Fractional Atomic Coordinates

The arrangement of the Compound I molecules in the N-5 form may additionally be characterized by the approximate fractional atomic coordinates listed in Table 7 below. The approximate coordinates in Table 7 will therefore vary according to the temperature at measurement. Statistical variations in these coordinates may also occur consistent with the reported error values.

TABLE 7
Fractional Atomic Parameters and Their Estimated Standard
Deviations for Form N-5 of Compound I at rt

| Atom | x | y | Z | B(iso) |
|---|---|---|---|---|
| F28 | 0.5084(3) | 0.3496(5) | 0.4693(4) | 14.2 |
| F29 | 0.5818(2) | 0.2684(6) | 0.4785(4) | 15.5 |
| F30 | 0.4944(4) | 0.2058(7) | 0.3904(4) | 17.8 |
| O13 | 0.34416(14) | 0.4911(3) | 0.54710(19) | 7.7 |
| O22 | 0.24705(12) | 0.8645(2) | 0.27830(17) | 6.2 |
| N1 | 0.43896(15) | 0.2260(3) | 0.5166(2) | 6.6 |
| N2 | 0.39445(17) | 0.1828(3) | 0.4358(3) | 7.4 |
| N12 | 0.5015(2) | 0.1036(5) | 0.6362(4) | 10.7 |
| N14I | 0.27825(14) | 0.4610(2) | 0.3976(2) | 5.6 |
| N23I | 0.19756(16) | 0.9578(3) | 0.3331(2) | 6.2 |
| C3I | 0.35044(19) | 0.2553(3) | 0.4110(3) | 6.6 |
| C4 | 0.36513(16) | 0.3423(3) | 0.4757(2) | 5.3 |
| C5 | 0.42321(19) | 0.3197(4) | 0.5429(3) | 6.4 |
| C6I | 0.4652(3) | 0.3809(7) | 0.6288(4) | 10.5 |
| C7 | 0.4949(2) | 0.1653(4) | 0.5669(4) | 7.8 |
| C8 | 0.5369(3) | 0.1743(5) | 0.5441(5) | 8.9 |
| C9I | 0.5898(3) | 0.1084(8) | 0.5993(7) | 12.1 |
| C10I | 0.5962(4) | 0.0454(8) | 0.6696(7) | 14.4 |
| C11I | 0.5529(5) | 0.0487(7) | 0.6860(6) | 12.4 |
| C13 | 0.32918(17) | 0.4369(3) | 0.4773(3) | 5.3 |
| C15 | 0.24071(16) | 0.5540(3) | 0.3933(2) | 5.0 |
| C16 | 0.19926(18) | 0.5365(3) | 0.4176(3) | 5.9 |
| C17I | 0.1655(2) | 0.6308(4) | 0.4123(3) | 6.7 |
| C18I | 0.17163(18) | 0.7375(3) | 0.3837(3) | 6.0 |
| C19 | 0.21304(15) | 0.7528(3) | 0.3577(2) | 4.9 |
| C20I | 0.24740(16) | 0.6597(3) | 0.3633(2) | 5.1 |
| C21I | 0.1909(2) | 0.4209(4) | 0.4497(4) | 7.8 |
| C22 | 0.22052(15) | 0.8632(3) | 0.3205(2) | 5.1 |
| C24I | 0.1945(2) | 1.0623(4) | 0.2863(3) | 7.0 |
| C25I | 0.1560(4) | 1.1535(6) | 0.2838(6) | 13.1 |
| C26I | 0.1384(3) | 1.0862(6) | 0.1987(4) | 8.8 |
| C27 | 0.5293(3) | 0.2485(7) | 0.4711(6) | 11.0 |

### C. Powder X-Ray Diffraction

X-ray powder diffraction (PXRD) data were obtained using the PXRD procedure described hereinabove. FIG. 10 and Table 8 show the PXRD data for the N-5 form of Compound I.

TABLE 8
Characteristic diffraction peak positions (degrees 2θ ± 0.1)@ RT,
based on a high quality pattern collected with a diffractometer
(CuKα) with a spinning capillary with 2θ calibrated
with a NIST other suitable standard.

| Peak # | 2-Theta (°) |
|---|---|
| 1 | 7.9 |
| 2 | 10.5 |
| 3 | 12.2 |
| 4 | 12.6 |
| 5 | 14.1 |
| 6 | 17.2 |
| 7 | 18.6 |
| 8 | 22.1 |

### D. Differential Scanning Calorimetry (DSC)

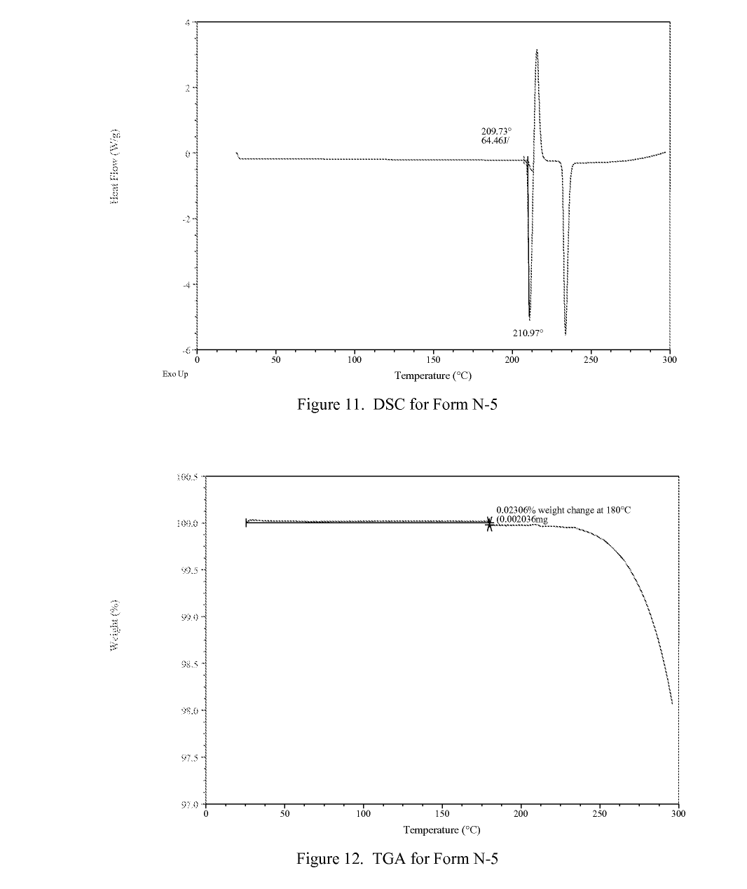

Differential scanning calorimetry was conducted using the procedure described above. FIG. 11 shows the DSC thermogram for the N-5 crystal form of Compound I, which was observed to have an endothermic transition with an onset in the range from about 208° C. to about 212° C.

### E. Thermogravimetric Analysis (TGA)

Thermogravimetric analysis was conducted using the procedure described above. FIG. 12 shows the TGA curve for the N-5 crystal form of Compound I, which has a negligible weight loss up to about 180° C.

### Example 5

### A. Single Crystal X-Ray Measurements

Following the above Single Crystal Data procedure, the approximate unit cell dimensions in Angstroms (Å), as measured at a sample temperature of 30° C., as well as the crystalline cell volume (V), space group (sg), molecules per unit cell, and crystal density for the N-6 form of Compound I are shown below.

Cell dimensions: a=9.2650(6) Å
b=39.143(5) Å
c=12.243(1) Å
α=90°
β=91.738(5)°
γ=90°
Volume=4438(1) Å³

Space group: P2₁/c

Molecules/unit cell (Z): 8

Density, calc g-cm⁻³: 1.327

The fractional atomic coordinates for the N-6 form of Compound I are tabulated in Table 9 hereinbelow.

### B. Fractional Atomic Coordinates

The arrangement of the Compound I molecules in the N-6 form may additionally be characterized by the approximate fractional atomic coordinates listed in Table 9 below. The approximate coordinates in Table 9 will therefore vary according to the temperature at measurement. Statistical variations in these coordinates may also occur consistent with the reported error values.

TABLE 9
Fractional Atomic Parameters and Their Estimated Standard
Deviations for Form N-6 of Compound I at 30° C.

| Atom | x | y | z | B(iso) |
|---|---|---|---|---|
| F28 | 0.2316(6) | 0.65551(14) | −0.1759(4) | 12.4 |
| F29 | 0.2653(9) | 0.68418(19) | −0.3171(5) | 13.2 |
| F30 | 0.440(2) | 0.6790(5) | −0.1890(17) | 13.3 |
| F32 | 0.420(6) | 0.6776(9) | −0.187(3) | 7.4 |
| F33 | 0.364(5) | 0.6916(10) | −0.287(4) | 18.8 |
| F58 | 0.7778(11) | 0.6860(2) | 0.8190(6) | 14.4 |
| F59 | 0.7383(16) | 0.6566(3) | 0.6752(9) | 13.7 |
| F60 | 0.9432(12) | 0.6836(3) | 0.6969(11) | 13.8 |
| F61 | 0.891(4) | 0.6989(10) | 0.794(4) | 13.7 |
| F62 | 0.912(5) | 0.6696(9) | 0.659(2) | 9.2 |
| F63 | 0.733(6) | 0.6615(17) | 0.723(4) | 7.7 |
| O13 | −0.0456(5) | 0.61656(10) | 0.1749(3) | 6.1 |
| O22 | −0.2498(5) | 0.48349(11) | 0.3170(4) | 8.1 |
| O43 | 0.4602(5) | 0.61410(9) | 0.3295(3) | 6.4 |
| O52 | 0.2466(5) | 0.48592(11) | 0.1759(4) | 8.1 |
| N1 | 0.2142(6) | 0.68747(13) | 0.0254(4) | 4.7 |
| N2 | 0.3471(6) | 0.67436(16) | 0.0504(5) | 7.6 |
| N12 | 0.1583(5) | 0.74404(18) | 0.0033(4) | 6.6 |
| N14I | 0.1599(5) | 0.59954(12) | 0.2585(4) | 5.1 |
| N23I | −0.0521(5) | 0.47751(15) | 0.2170(5) | 8.0 |
| N31 | 0.7160(6) | 0.68933(13) | 0.4794(4) | 5.8 |
| N32 | 0.8454(6) | 0.67998(16) | 0.4412(5) | 8.7 |
| N42 | 0.6460(6) | 0.74534(18) | 0.5001(4) | 7.3 |
| N44I | 0.6626(4) | 0.60026(12) | 0.2413(4) | 5.3 |
| N53I | 0.4449(5) | 0.47923(15) | 0.2829(5) | 6.9 |
| C3I | 0.3173(7) | 0.6481(2) | 0.1119(6) | 7.4 |
| C4 | 0.1696(7) | 0.64439(15) | 0.1291(4) | 4.8 |
| C5 | 0.1050(6) | 0.66941(17) | 0.0697(5) | 4.9 |
| C6I | −0.0468(7) | 0.68101(18) | 0.0506(6) | 8.6 |
| C7 | 0.2083(6) | 0.7162(2) | −0.0461(7) | 6.1 |
| C8 | 0.2429(7) | 0.7157(2) | −0.1545(8) | 7.8 |
| C9I | 0.2232(10) | 0.7459(3) | −0.2154(6) | 8.7 |
| C10I | 0.1702(10) | 0.7743(3) | −0.1645(11) | 10.3 |
| C11I | 0.1406(7) | 0.7719(2) | −0.0571(9) | 8.6 |
| C13 | 0.0853(8) | 0.61960(15) | 0.1871(5) | 5.3 |
| C15 | 0.0865(6) | 0.57589(16) | 0.3275(6) | 5.4 |
| C16 | 0.0677(6) | 0.58193(16) | 0.4383(6) | 6.5 |
| C17I | −0.0212(8) | 0.5599(2) | 0.4960(5) | 6.9 |
| C18I | −0.0885(6) | 0.53228(18) | 0.4446(7) | 6.9 |
| C19 | −0.0641(6) | 0.52505(17) | 0.3376(6) | 5.7 |
| C20I | 0.0229(6) | 0.54679(17) | 0.2783(5) | 5.1 |
| C21I | 0.1392(6) | 0.61239(16) | 0.4946(5) | 7.6 |
| C22 | −0.1315(8) | 0.49406(19) | 0.2890(6) | 6.9 |
| C24I | −0.1022(7) | 0.4462(2) | 0.1641(8) | 8.4 |
| C25I | −0.0034(13) | 0.4175(2) | 0.1575(10) | 13.5 |
| C26I | −0.0405(13) | 0.4364(3) | 0.0624(8) | 12.4 |
| C27 | 0.2919(15) | 0.6833(3) | −0.2077(10) | 10.8 |
| C33I | 0.8147(8) | 0.65315(19) | 0.3782(6) | 8.4 |
| C34 | 0.6686(7) | 0.64561(15) | 0.3758(5) | 5.0 |
| C35 | 0.6084(6) | 0.66945(18) | 0.4416(5) | 5.6 |
| C36I | 0.4567(6) | 0.6751(2) | 0.4715(6) | 11.6 |
| C37 | 0.7032(6) | 0.71808(19) | 0.5497(7) | 6.2 |
| C38 | 0.7411(7) | 0.7165(2) | 0.6566(8) | 7.4 |
| C39I | 0.7129(10) | 0.7462(3) | 0.7189(7) | 10.6 |
| C40I | 0.6541(11) | 0.7741(3) | 0.6702(11) | 11.5 |
| C41I | 0.6242(8) | 0.7731(2) | 0.5625(10) | 11.1 |
| C43 | 0.5899(7) | 0.61858(15) | 0.3147(5) | 4.9 |
| C45 | 0.5881(5) | 0.57581(17) | 0.1717(6) | 5.0 |
| C46 | 0.5719(6) | 0.58179(16) | 0.0596(6) | 5.1 |
| C47I | 0.4834(8) | 0.5596(2) | 0.0002(5) | 7.2 |
| C48I | 0.4149(6) | 0.53258(18) | 0.0494(7) | 7.0 |
| C49 | 0.4377(6) | 0.52547(17) | 0.1585(7) | 5.2 |
| C50I | 0.5243(6) | 0.54778(17) | 0.2208(5) | 4.8 |
| C51I | 0.6448(6) | 0.61186(17) | 0.0074(5) | 6.6 |
| C52 | 0.3665(8) | 0.49540(18) | 0.2062(6) | 6.2 |
| C54I | 0.3935(8) | 0.4498(3) | 0.3368(8) | 9.9 |
| C55I | 0.4780(14) | 0.4187(3) | 0.3387(11) | 13.6 |
| C56I | 0.4635(13) | 0.4376(3) | 0.4340(8) | 13.6 |
| C57 | 0.8035(16) | 0.6844(4) | 0.7102(10) | 11.9 |

### C. Powder X-Ray Diffraction

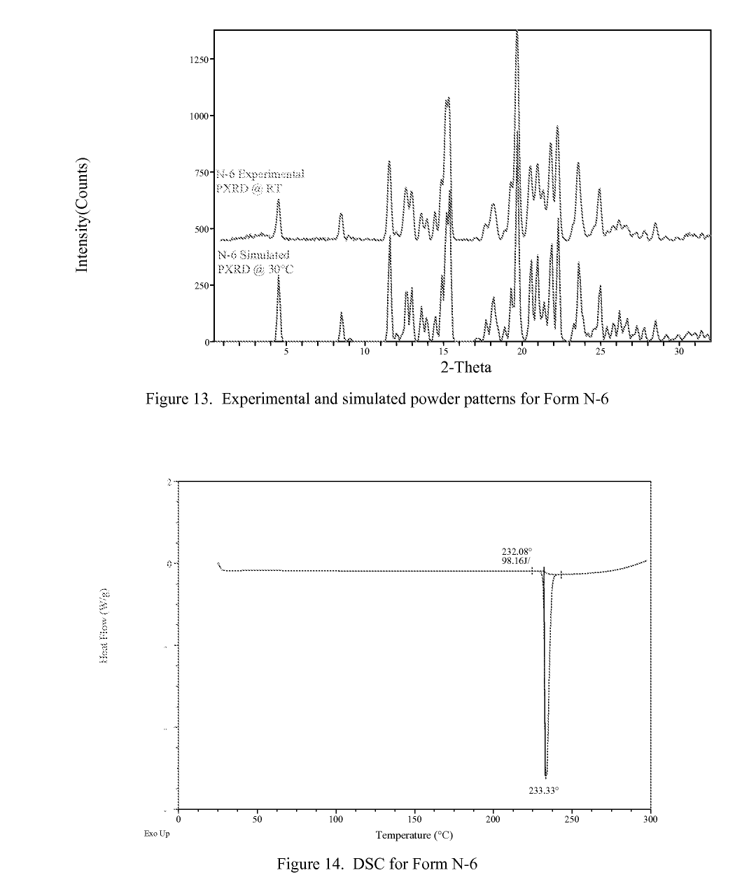

X-ray powder diffraction (PXRD) data were obtained using the PXRD procedure described hereinabove. FIG. 13 and Table 10 show the PXRD data for the N-6 form of Compound I.

TABLE 10
Characteristic diffraction peak positions (degrees 2θ ± 0.1)@ RT,
based on a high quality pattern collected with a diffractometer
(CuKα) with a spinning capillary with 2θ calibrated
with a NIST other suitable standard.

| Peak # | 2-Theta (°) |
|---|---|
| 1 | 4.5 |
| 2 | 8.5 |
| 3 | 11.6 |
| 4 | 12.6 |
| 5 | 13.0 |
| 6 | 19.7 |
| 7 | 21.0 |
| 8 | 22.3 |

### D. Differential Scanning Calorimetry (DSC)

Differential scanning calorimetry was conducted using the procedure described above. FIG. 14 shows the DSC thermogram for the N-6 crystal form of Compound I, which was observed to have an endothermic transition with an onset in the range from about 229° C. to about 233° C.

### E. Thermogravimetric Analysis (TGA)

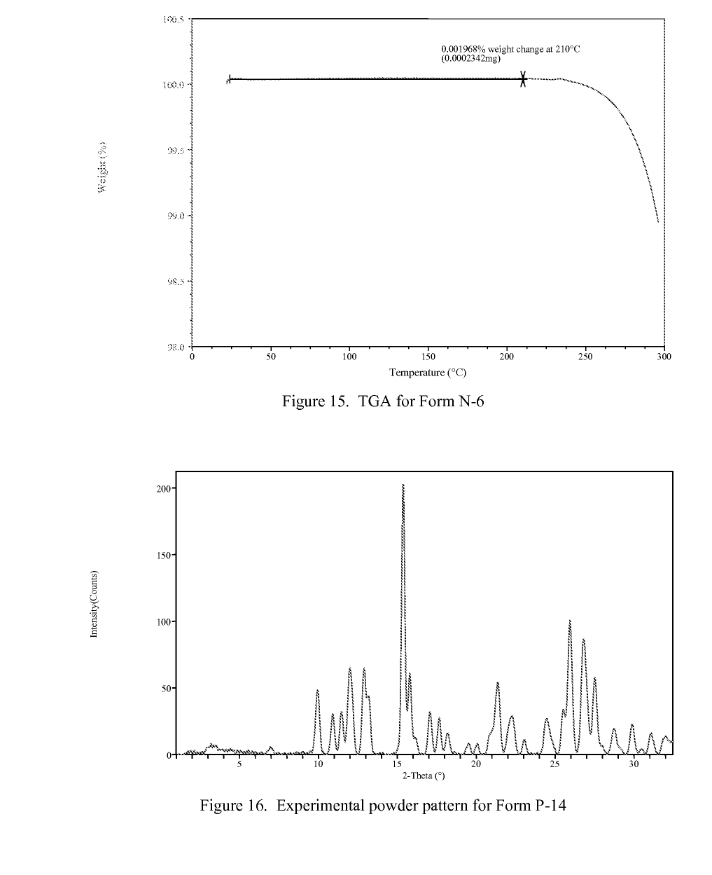

Thermogravimetric analysis was conducted using the procedure described above. FIG. 15 shows the TGA curve for the N-6 crystal form of Compound I, which has a negligible weight loss up to about 210° C.

### Example 6

### A. Powder X-Ray Diffraction

X-ray powder diffraction (PXRD) data were obtained for the P-14 crystalline form of Compound I using the PXRD procedure described hereinabove and is shown in FIG. 16.

### B. Differential Scanning Calorimetry (DSC)

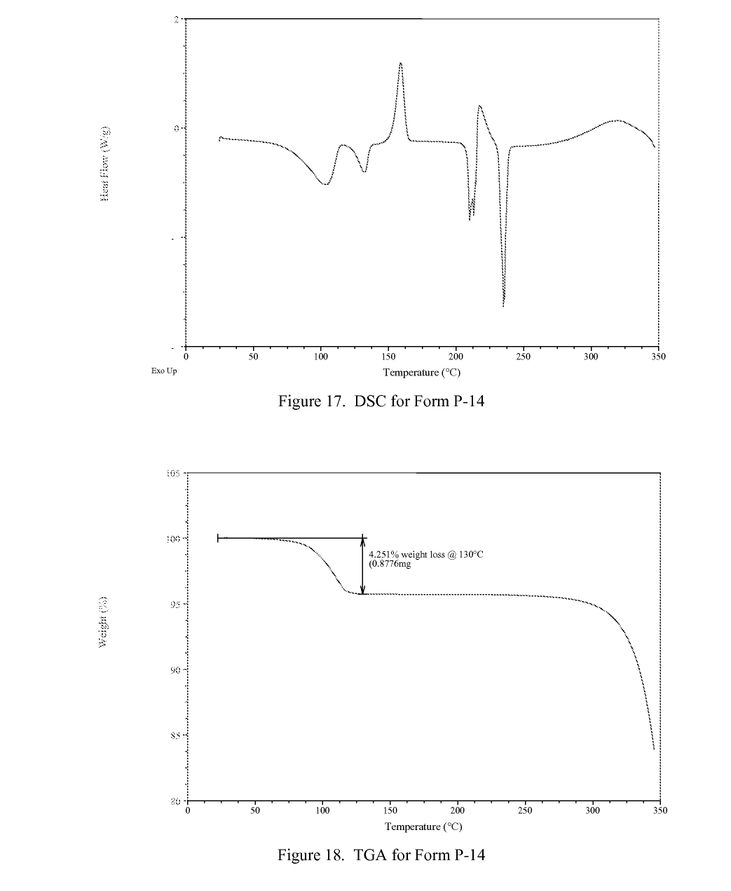

Differential scanning calorimetry was conducted using the procedure described above. FIG. 17 shows the DSC thermogram for the P-14 crystal form of Compound I.

### C. Thermogravimetric Analysis (TGA)

Thermogravimetric analysis was conducted using the procedure described above. FIG. 18 shows the TGA curve for the P-14 crystal form of Compound I.

### Example 7

### A. Single Crystal X-Ray Measurements

Following the above Single Crystal Data procedure, the approximate unit cell dimensions in Angstroms (A), as measured at a sample temperature of −70° C., as well as the crystalline cell volume (V), space group (sg), molecules per unit cell, and crystal density for the AN-3 form of Compound I are shown below.

Cell dimensions: a=7.599(2) Å
b=11.123(5) Å
c=14.246(4) Å
α=103.05(3)°
β=93.72(2)°
γ=102.24(3)°
Volume=1138(1) Å³

Space group: P-1

Molecules/unit cell (Z): 2

Density, calc g-cm⁻³: 1.413

The fractional atomic coordinates for the AN-3 form of Compound I are tabulated in Table 11 hereinbelow.

### B. Fractional Atomic Coordinates

The arrangement of the Compound I molecules in the AN-3 form may additionally be characterized by the approximate fractional atomic coordinates listed in Table 11 below. The approximate coordinates in Table 11 will therefore vary according to the temperature at measurement. Statistical variations in these coordinates may also occur consistent with the reported error values.

TABLE 11
Fractional Atomic Parameters and Their Estimated Standard
Deviations for Form AN-3 of Compound I at −70° C.

| Atom | x | y | z | B(iso) |
|---|---|---|---|---|
| F28 | 0.6378(3) | −0.0748(2) | 0.28823(14) | 4.0 |
| F29 | 0.6000(2) | 0.08664(17) | 0.39324(14) | 3.8 |
| F30 | 0.6422(3) | −0.07698(18) | 0.43744(12) | 3.5 |
| O13 | 0.1453(3) | 0.43895(19) | 0.31245(14) | 3.2 |
| O22 | 0.6244(3) | 0.7175(2) | 0.00939(16) | 3.3 |
| N1 | 0.2675(3) | 0.0772(2) | 0.29520(16) | 2.2 |
| N2 | 0.3750(4) | 0.0806(2) | 0.22094(18) | 2.5 |
| N12 | 0.0495(3) | −0.0814(2) | 0.32757(18) | 2.5 |
| N14I | 0.2220(4) | 0.4001(2) | 0.15851(18) | 2.3 |
| N23I | 0.5578(3) | 0.9022(2) | 0.08243(18) | 2.6 |
| N99 | 0.6078(5) | 0.5954(4) | 0.3880(3) | 7.1 |
| C3I | 0.3648(4) | 0.1881(3) | 0.1976(2) | 2.6 |
| C4 | 0.2554(4) | 0.2544(3) | 0.2540(2) | 2.1 |
| C5 | 0.1938(4) | 0.1796(3) | 0.31695(19) | 2.1 |
| C6I | 0.0741(5) | 0.2003(3) | 0.3953(2) | 2.9 |
| C7 | 0.2255(4) | −0.0373(3) | 0.3274(2) | 2.1 |
| C8 | 0.3581(4) | −0.0961(3) | 0.3529(2) | 2.2 |
| C9I | 0.2965(5) | −0.2167(3) | 0.3700(2) | 2.7 |
| C10I | 0.1169(4) | −0.2647(3) | 0.3676(2) | 2.7 |
| C11I | −0.0035(5) | −0.1940(3) | 0.3494(2) | 2.7 |
| C13 | 0.2029(4) | 0.3730(3) | 0.2458(2) | 2.4 |
| C15 | 0.2062(4) | 0.5181(3) | 0.1377(2) | 2.2 |
| C16 | 0.0434(4) | 0.5560(3) | 0.1372(2) | 2.4 |
| C17I | 0.0441(4) | 0.6723(3) | 0.1141(2) | 2.5 |
| C18I | 0.1958(4) | 0.7457(3) | 0.0917(2) | 2.4 |
| C19 | 0.3568(4) | 0.7041(3) | 0.0883(2) | 2.5 |
| C20I | 0.3583(4) | 0.5890(3) | 0.1106(2) | 2.5 |
| C21I | −0.1294(4) | 0.4791(3) | 0.1578(2) | 2.3 |
| C22 | 0.5223(4) | 0.7754(3) | 0.0573(2) | 2.3 |
| C24I | 0.7186(4) | 0.9797(3) | 0.0611(2) | 2.7 |
| C25I | 0.8945(5) | 0.9948(3) | 0.1190(2) | 3.3 |
| C26I | 0.8017(5) | 1.1035(3) | 0.1314(3) | 3.4 |
| C27 | 0.5566(4) | −0.0402(3) | 0.3672(2) | 2.7 |
| C97I | 0.7155(8) | 0.4085(5) | 0.4287(4) | 6.9 |
| C98 | 0.6527(5) | 0.5125(4) | 0.4055(3) | 4.5 |

### C. Powder X-Ray Diffraction

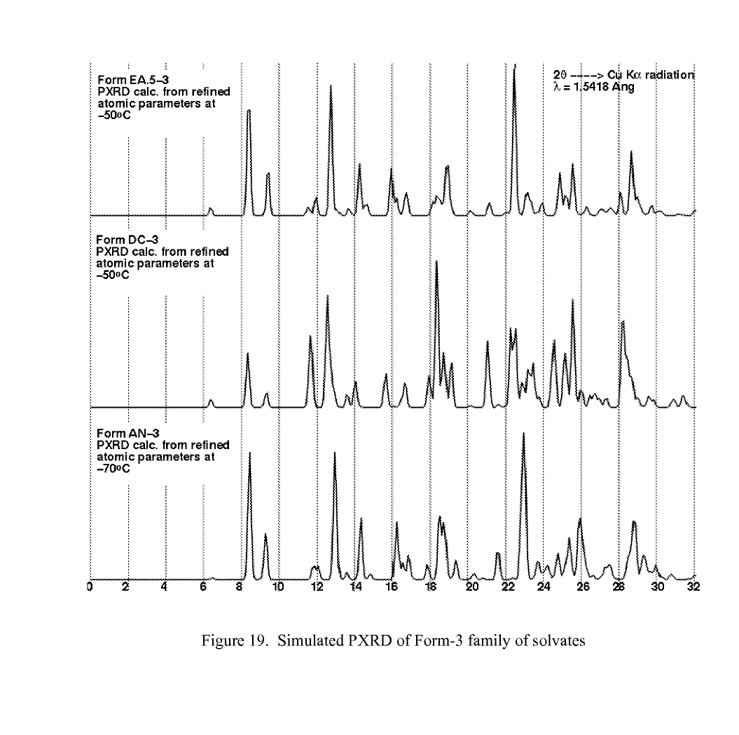

X-ray powder diffraction (PXRD) data were obtained for the AN-3 crystalline form of Compound I using the PXRD procedure described hereinabove. FIG. 19 shows simulated powder x-ray diffraction patterns (CuKalpha radiation) from the form-3 family of solvates (EA.5-3 ( at −50° C.), DC-3 (at −50° C.), and AN-3 (at −70° C.) of Compound I. The type-3 family includes EA.5-3 (EtOAc disordered about the center), DC-3 (CH₂Cl₂ disordered about the center) and AN-3 (ordered in the void space). The propyl acetate solvate, form PA-3, is isostructural but has not been determined by single crystal analysis. The void space is ˜80 A³.

### Example 8

### A. Single Crystal X-Ray Measurements

Following the above Single Crystal Data procedure, the approximate unit cell dimensions in Angstroms (Å), as measured at a sample temperature of −50° C., as well as the crystalline cell volume (V), space group (sg), molecules per unit cell, and crystal density for the E-8 form of Compound I are shown below.

Cell dimensions: a=7.689(2) Å
b=28.698(1) Å
c=11.086(1) Å
α=90°
β=100.37(2)°
γ=90°
Volume=2406.3(4) Å³

Space group: P2₁/a

Molecules/unit cell (Z): 4

Density, calc g-cm⁻³: 1.351

The fractional atomic coordinates for the E-8 form of Compound I are tabulated in Table 12 hereinbelow.

### B. Fractional Atomic Coordinates

The arrangement of the Compound I molecules in the E-8 form may additionally be characterized by the approximate fractional atomic coordinates listed in Table 12 below. The approximate coordinates in Table 12 will therefore vary according to the temperature at measurement. Statistical variations in these coordinates may also occur consistent with the reported error values.

TABLE 12
Fractional Atomic Parameters and Their Estimated Standard Deviations
for Form E-8 of Compound I at −50° C.

| | | | | | | | Occu- | |
|---|---|---|---|---|---|---|---|---|
| Atom | x | | y | | Z | | pancy | B (iso) |
| F29 | 0.5009 | (12) | 0.3052 | (3) | 1.0090 | (10) | | 9.8 |
| F28 | 0.4221 | (12 | 0.3513 | (3) | 1.1407 | (9 | | 9.6 |
| F30 | 0.4507 | (10 | 0.2798 | (3) | 1.1819 | (9 | | 10.6 |
| O13 | 0.8967 | (14 | 0.3524 | (3) | 0.6430 | (9 | | 8.0 |
| O22 | 0.3643 | (13 | 0.4917 | (3) | 0.2813 | (8 | | 6.4 |
| N1 | 0.7989 | (15 | 0.3581 | (4) | 1.0107 | (9 | | 5.1 |
| N2 | 0.6826 | (17 | 0.3933 | (4) | 0.9903 | (9) | | 6.5 |
| N12 | 1.0092 | (10) | 0.3414 | (4) | 1.1804 | (13 | | 6.6 |
| N14 | 0.8054 | (13) | 0.4281 | (4) | 0.6442 | (9) | | 5.5 |
| N23 | 0.4527 | (14) | 0.4563 | (4) | 0.1195 | (10) | | 5.4 |
| C3 | 0.6825 | (18) | 0.4082 | (4) | 0.8727 | (9) | | 6.1 |
| C4 | 0.7991 | (20) | 0.3812 | (5) | 0.8206 | (11) | | 5.5 |
| C5 | 0.8701 | (19) | 0.3499 | (5) | 0.9072 | (14 | | 6.3 |
| C6 | 1.0098 | (20) | 0.3121 | (5 | 0.9092 | (10 | | 7.8 |
| C7 | 0.8405 | (20) | 0.3395 | (5) | 1.1279 | (13) | | 5.7 |
| C8 | 0.7130 | (26) | 0.3217 | (5) | 1.1905 | (18) | | 6.3 |
| C9 | 0.7524 | (27 | 0.3074 | (6) | 1.3082 | (19) | | 7.8 |
| C10 | 0.9240 | (41) | 0.3116 | (6) | 1.3609 | (16) | | 9.8 |
| C11 | 1.0659 | (24) | 0.3273 | (6) | 1.2923 | (17) | | 8.3 |
| C27 | 0.5346 | (34) | 0.3153 | (5) | 1.1333 | (18) | | 8.0 |
| C13 | 0.8420 | (20) | 0.3873 | (5) | 0.6958 | (12) | | 5.8 |
| C15 | 0.8156 | (12) | 0.4362 | (4) | 0.5130 | (12) | | 6.6 |
| C20 | 0.6516 | (21) | 0.4482 | (4) | 0.4411 | (13) | | 5.8 |
| C19 | 0.6431 | (22) | 0.4561 | (4) | 0.3125 | (13) | | 6.0 |
| C18 | 0.8120 | (21) | 0.4520 | (4) | 0.2677 | (12) | | 5.9 |
| C17 | 0.9663 | (20) | 0.4452 | (4) | 0.3497 | (12) | | 6.0 |
| C16 | 0.9731 | (18) | 0.4346 | (4) | 0.4699 | (12) | | 5.0 |
| C71 | 1.1410 | (20) | 0.4265 | (4) | 0.5559 | (12) | | 7.1 |
| C22 | 0.4738 | (21) | 0.4691 | (4) | 0.2386 | (12) | | 5.5 |
| C24 | 0.2984 | (20) | 0.4678 | (2) | 0.0358 | (12) | | 5.6 |
| C25 | 0.1280 | (19) | 0.4398 | (2) | 0.0377 | (12) | | 7.2 |
| C26 | 0.2379 | (20) | 0.4354 | (4) | −0.0698 | (13) | | 7.5 |
| O99 | 0.6477 | (4) | 0.2786 | (3) | 0.5788 | (6) | | 26. |
| C98 | 0.5168 | | 0.2765 | | 0.6619 | | 0.5 | 15. |
| C97 | 0.4109 | | 0.3090 | | 0.6678 | | 0.5 | 12 |
| C96 | 0.4257 | | 0.2992 | | 0.5351 | | 0.5 | 18 |

Occupancies are 1. unless otherwise indicated

### Example 9

### A. Single Crystal X-Ray Measurements

Following the above Single Crystal Data procedure, the approximate unit cell dimensions in Angstroms (Å), as measured at a sample temperature of −50° C., as well as the crystalline cell volume (V), space group (sg), molecules per unit cell, and crystal density for the EA.5-3 form of Compound I are shown below.

Cell dimensions: a=7.655(1) Å
b=11.025(1) Å
c=14.362(2) Å
α=100.89(1)°
β=95.42(1)°
γ=100.68(1)°
Volume=1159.0(5) Å³

Space group: P-1

Molecules/unit cell (Z): 2

Density, calc g-cm⁻³: 1.400

The fractional atomic coordinates for the EA.5-3 form of Compound I are tabulated in Table 13 hereinbelow.

### B. Fractional Atomic Coordinates

The arrangement of the Compound I molecules in the EA.5-3 form may additionally be characterized by the approximate fractional atomic coordinates listed in Table 13 below. The approximate coordinates in Table 13 will therefore vary according to the temperature at measurement. Statistical variations in these coordinates may also occur consistent with the reported error values.

TABLE 13
Fractional Atomic Parameters and Their Estimated Standard
Deviations for Form EA.5-3 of Compound I at −50° C.

| | | | | | | | Occu- | | |
|---|---|---|---|---|---|---|---|---|---|
| Atom | x | | y | | Z | | pancy | B (A2) | |
| F28 | 0.6435 | (5) | −0.0843 | (4) | 0.2926 | (3) | | 4.4 | (1) |
| F29 | 0.6023 | (5) | 0.0750 | (4) | 0.3927 | (3) | | 4.2 | (1) |
| F30 | 0.6512 | (5) | −0.0891 | (4) | 0.4412 | (3) | | 4.18 | (9) |
| O13 | 0.1631 | (6) | 0.4350 | (4) | 0.3059 | (3) | | 3.1 | (1) |
| O22 | 0.6312 | (6) | 0.7210 | (4) | 0.0128 | (3) | | 2.86 | (9) |
| N1 | 0.2716 | (6) | 0.0666 | (4) | 0.2928 | (3) | | 1.8 | (1) |
| N2 | 0.3757 | (6) | 0.0680 | (4) | 0.2197 | (3) | | 2.0 | (1) |
| N14 | 0.2286 | (6) | 0.3962 | (4) | 0.1522 | (3) | | 2.0 | (1) |
| N23 | 0.5642 | (6) | 0.9034 | (4) | 0.0867 | (3) | | 1.8 | (1) |
| C3 | 0.3655 | (8) | 0.1770 | (5) | 0.1935 | (4) | | 2.0 | (1) |
| C4 | 0.2587 | (7) | 0.2440 | (5) | 0.2479 | (4) | | 1.7 | (1) |
| C5 | 0.1985 | (7) | 0.1699 | (5) | 0.3123 | (4) | | 1.7 | (1) |
| C6 | 0.0820 | (9) | 0.1922 | (6) | 0.3886 | (4) | | 2.8 | (1) |
| C7 | 0.2340 | (8) | −0.0475 | (5) | 0.3279 | (4) | | 1.8 | (1) |
| C8 | 0.3686 | (8) | −0.1062 | (5) | 0.3562 | (4) | | 2.1 | (1) |
| C9 | 0.3143 | (9) | −0.2260 | (5) | 0.3759 | (4) | | 2.8 | (1) |
| C10 | 0.1342 | (9) | −0.2754 | (6) | 0.3725 | (5) | | 3.1 | (2) |
| C11 | 0.0125 | (9) | −0.2048 | (6) | 0.3515 | (5) | | 2.9 | (1) |
| N12 | 0.0590 | (7) | −0.0930 | (4) | 0.3269 | (4) | | 2.3 | (1) |
| C13 | 0.2124 | (8) | 0.3663 | (5) | 0.2395 | (4) | | 2.0 | (1) |
| C15 | 0.2145 | (8) | 0.5177 | (5) | 0.1329 | (4) | | 1.9 | (1) |
| C16 | 0.0553 | (8) | 0.5614 | (5) | 0.1332 | (4) | | 1.7 | (1) |
| C17 | 0.0560 | (8) | 0.6793 | (5) | 0.1130 | (4) | | 2.3 | (1) |
| C18 | 0.2106 | (8) | 0.7532 | (5) | 0.0929 | (4) | | 2.0 | (1) |
| C19 | 0.3663 | (8) | 0.7069 | (5) | 0.0888 | (4) | | 1.8 | (1) |
| C20 | 0.3684 | (8) | 0.5879 | (5) | 0.1080 | (4) | | 1.9 | (1) |
| C21 | −0.1181 | (8) | 0.4834 | (6) | 0.1506 | (5) | | 2.6 | (1) |
| C22 | 0.5322 | (8) | 0.7770 | (5) | 0.0598 | (4) | | 1.8 | (1) |
| C24 | 0.7200 | (8) | 0.9819 | (5) | 0.0640 | (4) | | 2.2 | (1) |
| C25 | 0.8974 | (8) | 0.9956 | (6) | 0.1221 | (5) | | 2.9 | (1) |
| C26 | 0.7997 | (9) | 1.1027 | (6) | 0.1326 | (5) | | 2.9 | (2) |
| C27 | 0.5647 | (9) | −0.0505 | (6) | 0.3700 | (5) | | 3.1 | (2) |
| C96 | 0.725 | (2) | 0.400 | (2) | 0.396 | (1) | 0.48 | 6.2 | |
| C97 | 0.643 | (1) | 0.587 | (1) | 0.4058 | (8) | 0.76 | 5.4 | |
| C98 | 0.637 | (2) | 0.488 | (1) | 0.4223 | (9) | 0.69 | 6.5 | |
| C99 | 0.538 | (1) | 0.4582 | (7) | 0.4866 | (6) | 1. | 5.6 | |

Occupancies are 1. unless otherwise indicated

### C. Powder X-Ray Diffraction

X-ray powder diffraction (PXRD) data were obtained for the EA.5-3 form of Compound I using the PXRD procedure described hereinabove. FIG. 19 shows simulated (calculated at −50° C.) powder x-ray diffraction patterns (CuKα λ=1.5418 Å) from the form-3 family of solvates (EA.5-3, DC-3, AN-3 and PA-3) of Compound I.

### Example 10

### A. Single Crystal X-Ray Measurements

Following the above Single Crystal Data procedure, the approximate unit cell dimensions in Angstroms (Å), as measured at a sample temperature of −50° C., as well as the crystalline cell volume (V), space group (sg), molecules per unit cell, and crystal density for the IPA-10 form of Compound I are shown below.

Cell dimensions: a=10.695(3) Å
b=15.105(4) Å
c=16.518(4) Å
α=73.67(2)°
β=89.62(2)°
γ=82.14(2)°
Volume=2535(2) Å³

Space group: P-1

Molecules/unit cell (Z): 8

Density, calc g-cm⁻³: 1.361

The fractional atomic coordinates for the IPA-10 form of Compound I are tabulated in Table 14 hereinbelow.

### B. Fractional Atomic Coordinates

The arrangement of the Compound I molecules in the IPA-10 form may additionally be characterized by the approximate fractional atomic coordinates listed in Table 14 below. The approximate coordinates in Table 14 will therefore vary according to the temperature at measurement. Statistical variations in these coordinates may also occur consistent with the reported error values.

TABLE 14
Fractional Atomic Parameters and Their Estimated Standard
Deviations for Form IPA-10 of Compound I at −50° C.

| Atom | x | y | z | B(iso) |
|---|---|---|---|---|
| F28 | 0.730(2) | 0.699(1) | −0.1801(9) | 6.9(4) |
| F29 | 0.607(2) | 0.609(1) | −0.1070(9) | 6.6(4) |
| F30 | 0.797(2) | 0.556(1) | −0.131(1) | 7.8(5) |
| O13 | 0.213(1) | 0.7207(9) | 0.1011(8) | 2.7(3) |
| O22 | −0.214(1) | 1.0133(9) | 0.1780(8) | 2.8(3) |
| N1 | 0.578(2) | 0.723(1) | 0.0009(9) | 2.2(4) |
| N2 | 0.551(2) | 0.797(1) | −0.072(1) | 2.9(4) |
| N12 | 0.747(2) | 0.691(1) | 0.0922(9) | 2.3(4) |
| N14 | 0.162(2) | 0.840(1) | −0.0197(9) | 2.4(4) |
| N23 | −0.072(2) | 0.889(1) | 0.247(1) | 3.1(4) |
| C3 | 0.427(2) | 0.820(1) | −0.068(1) | 2.5(5) |
| C4 | 0.375(2) | 0.765(1) | 0.004(1) | 1.4(4) |
| C5 | 0.478(2) | 0.699(1) | 0.043(1) | 2.2(5) |
| C6 | 0.482(2) | 0.621(2) | 0.126(1) | 3.9(6) |
| C7 | 0.709(2) | 0.684(1) | 0.018(1) | 1.6(4) |
| C8 | 0.778(2) | 0.644(1) | −0.036(1) | 1.5(4) |
| C9 | 0.907(2) | 0.619(1) | −0.015(1) | 2.5(5) |
| C10 | 0.954(2) | 0.629(1) | 0.057(1) | 3.3(5) |
| C11 | 0.875(2) | 0.665(1) | 0.109(1) | 3.2(5) |
| C13 | 0.246(2) | 0.771(1) | 0.034(1) | 2.0(4) |
| C15 | 0.034(2) | 0.864(1) | 0.003(1) | 1.4(4) |
| C16 | −0.064(2) | 0.875(1) | −0.055(1) | 1.9(4) |
| C17 | −0.186(2) | 0.908(1) | −0.037(1) | 2.6(5) |
| C18 | −0.208(2) | 0.929(1) | 0.040(1) | 2.1(5) |
| C19 | −0.112(2) | 0.916(1) | 0.097(1) | 1.8(4) |
| C20 | 0.012(2) | 0.887(1) | 0.078(1) | 1.0(4) |
| C21 | −0.045(2) | 0.853(1) | −0.139(1) | 2.8(5) |
| C22 | −0.129(2) | 0.943(1) | 0.176(1) | 2.6(5) |
| C24 | −0.098(2) | 0.905(2) | 0.333(1) | 3.9(6) |
| C25 | −0.006(2) | 0.856(2) | 0.398(1) | 4.5(6) |
| C27 | 0.718(2) | 0.628(2) | −0.113(1) | 4.7(6) |
| C26 | −0.009(3) | 0.957(2) | 0.355(2) | 7.8(9) |
| O99 | 0.086(1) | 0.7099(9) | 0.2470(8) | 3.2(3) |
| C96 | 0.271(3) | 0.660(2) | 0.337(2) | 5.5(7) |
| C97 | 0.171(2) | 0.547(2) | 0.284(1) | 4.9(7) |
| C98 | 0.156(2) | 0.635(2) | 0.316(1) | 4.4(6) |
| F58 | 0.783(1) | 0.300(1) | 0.1624(9) | 6.1(4) |
| F59 | 0.708(1) | 0.442(1) | 0.1360(9) | 6.4(4) |
| F60 | 0.892(1) | 0.394(1) | 0.1922(9) | 6.7(4) |
| O43 | 1.280(1) | 0.2792(9) | 0.4593(8) | 2.6(3) |
| O52 | 1.715(1) | −0.0137(9) | 0.6785(8) | 2.3(3) |
| N31 | 0.920(2) | 0.279(1) | 0.360(1) | 2.6(4) |
| N32 | 0.950(2) | 0.208(1) | 0.319(1) | 2.8(4) |
| N42 | 0.742(2) | 0.306(1) | 0.4316(9) | 2.1(4) |
| N44 | 1.336(2) | 0.161(1) | 0.3994(9) | 2.0(4) |
| N53 | 1.575(2) | 0.110(1) | 0.6880(9) | 2.3(4) |
| C33 | 1.072(2) | 0.187(1) | 0.337(1) | 2.7(5) |
| C34 | 1.121(2) | 0.238(1) | 0.384(1) | 1.8(4) |
| C35 | 1.016(2) | 0.301(1) | 0.394(1) | 1.8(4) |
| C36 | 1.007(2) | 0.373(1) | 0.441(1) | 3.4(5) |
| C37 | 0.785(2) | 0.315(1) | 0.355(1) | 1.7(4) |
| C38 | 0.720(2) | 0.354(1) | 0.279(1) | 1.7(4) |
| C39 | 0.589(2) | 0.379(1) | 0.285(1) | 2.6(5) |
| C40 | 0.539(2) | 0.367(1) | 0.362(1) | 3.1(5) |
| C41 | 0.613(2) | 0.331(1) | 0.432(1) | 3.0(5) |
| C43 | 1.248(2) | 0.228(1) | 0.417(1) | 2.3(5) |
| C45 | 1.462(2) | 0.135(1) | 0.433(1) | 1.4(4) |
| C46 | 1.560(2) | 0.127(1) | 0.376(1) | 1.4(4) |
| C47 | 1.679(2) | 0.092(1) | 0.412(1) | 2.3(5) |
| C48 | 1.704(2) | 0.070(1) | 0.499(1) | 2.3(5) |
| C49 | 1.610(2) | 0.083(1) | 0.551(1) | 1.7(4) |
| C50 | 1.486(2) | 0.114(1) | 0.520(1) | 1.3(4) |
| C51 | 1.534(2) | 0.150(1) | 0.281(1) | 2.2(5) |
| C52 | 1.633(2) | 0.054(1) | 0.643(1) | 2.8(5) |
| C54 | 1.607(2) | 0.092(2) | 0.781(1) | 4.3(6) |
| C55 | 1.525(2) | 0.147(2) | 0.823(1) | 4.6(6) |
| C57 | 0.786(2) | 0.371(2) | 0.196(1) | 4.5(6) |
| C56 | 1.512(3) | 0.049(2) | 0.828(2) | 9(1) |
| O89 | 1.411(1) | 0.2834(9) | 0.6019(8) | 3.4(3) |
| C86 | 1.226(3) | 0.331(2) | 0.666(2) | 7.5(9) |
| C87 | 1.326(3) | 0.444(2) | 0.559(2) | 6.6(8) |
| C88 | 1.345(3) | 0.357(2) | 0.636(2) | 5.6(7) |

### Example 11

### A. Single Crystal X-Ray Measurements

Following the above Single Crystal Data procedure, the approximate unit cell dimensions in Angstroms (Å), as measured at a sample temperature of −50° C., as well as the crystalline cell volume (V), space group (sg), molecules per unit cell, and crystal density for the SA-9 form of Compound I are shown below.

Cell dimensions: a=10.897(2) Å
b=11.662(2) Å
c=12.117(2) Å
α=90.53(1)°
β=106.265(8)°
γ=105.14(1)°
Volume=1421.2(5) Å³

Space group: P-1

Molecules/unit cell (Z): 2

Density, calc g-cm⁻³: 1.289

The fractional atomic coordinates for the SA-9 form of Compound I are tabulated in Table 15 hereinbelow.

### B. Fractional Atomic Coordinates

The arrangement of the Compound I molecules in the SA-9 form may additionally be characterized by the approximate fractional atomic coordinates listed in Table 15 below. The approximate coordinates in Table 15 will therefore vary according to the temperature at measurement. Statistical variations in these coordinates may also occur consistent with the reported error values.

TABLE 15
Fractional Atomic Parameters and Their Estimated Standard
Deviations for Form SA-9 of Compound I at −50° C.

| | | | | | | | Occu- | B |
|---|---|---|---|---|---|---|---|---|
| Atom | X | | y | | z | | pancy | (iso) |
| F28 | 0.1979 | (7) | 0.3176 | ( ) | 0.0223 | (4) | | 9.8 |
| F29 | 0.3414 | ( ) | 0.3331 | (4) | 0.1833 | (2) | | 10.5 |
| F30 | 0.3118 | (4) | 0.1936 | (4) | 0.0608 | (4) | | 10. |
| O13 | 0.2878 | (4) | 0.6654 | (4) | 0.4529 | (4) | | 4.9 |
| O22 | 0.5360 | (4) | 1.0686 | (4) | 0.1750 | (4) | | 5.4 |
| O98 | 0.1371 | (4) | 0.7708 | (4) | 0.0540 | (4) | | 5.3 |
| O99 | 0.3251 | (4) | 0.9336 | (4) | −0.0106 | (4) | | 6.9 |
| N1 | 0.0950 | (4) | 0.3510 | (4) | 0.2252 | (4) | | 4.1 |
| N2 | 0.0228 | (4) | 0.4010 | (4) | 0.1370 | (4) | | 4.4 |
| N12 | −0.0115 | (4) | 0.1583 | (4) | 0.2573 | (4) | | 5.0 |
| N14 | 0.2035 | (4) | 0.7528 | (4) | 0.2930 | (4) | | 3.8 |
| N23 | 0.6286 | (4) | 1.2142 | (4) | 0.3212 | (4) | | 4.0 |
| C3 | 0.0586 | (4) | 0.5146 | (4) | 0.1732 | (4) | | 4.1 |
| C4 | 0.1535 | (4) | 0.5403 | (4) | 0.2839 | (4) | | 3.6 |
| C5 | 0.1733 | (4) | 0.4308 | (4) | 0.3152 | (4) | | 4.2 |
| C6 | 0.2583 | (4) | 0.3961 | (4) | 0.4178 | (4) | | 6.8 |
| C7 | 0.0774 | (4) | 0.2249 | (4) | 0.2131 | (4) | | 3.9 |
| C8 | 0.1457 | (4) | 0.1777 | (4) | 0.1505 | (4) | | 4.4 |
| C9 | 0.1162 | (4) | 0.0545 | (4) | 0.1321 | (4) | | 5.0 |
| C10 | 0.0231 | (4) | −0.0147 | (4) | 0.1787 | (4) | | 5.6 |
| C11 | −0.0377 | (4) | 0.0404 | (4) | 0.2411 | (4) | | 5.6 |
| C13 | 0.2198 | (4) | 0.6548 | (4) | 0.3505 | (4) | | 3.7 |
| C15 | 0.2729 | (4) | 0.8717 | (4) | 0.3392 | (4) | | 3.3 |
| C16 | 0.2387 | (4) | 0.9292 | (4) | 0.4236 | (4) | | 3.6 |
| C17 | 0.3089 | (4) | 1.0470 | (4) | 0.4604 | (4) | | 4.0 |
| C18 | 0.4078 | (4) | 1.1095 | (4) | 0.4154 | (4) | | 3.8 |
| C19 | 0.4377 | (4) | 1.0526 | (4) | 0.3281 | (4) | | 3.4 |
| C20 | 0.3698 | (4) | 0.9335 | (4) | 0.2919 | (4) | | 3.7 |
| C21 | 0.1243 | (4) | 0.8667 | (4) | 0.4696 | (4) | | 4.9 |
| C22 | 0.5377 | (4) | 1.1139 | (4) | 0.2695 | (4) | | 4.4 |
| C24 | 0.7281 | (4) | 1.2756 | (4) | 0.2705 | (4) | | 1.3 |
| C25 | 0.6942 | (4) | 1.3567 | (4) | 0.1796 | (4) | | 6.7 |
| C26 | 0.7792 | (4) | 1.4051 | (4) | 0.2977 | (4) | | 7.2 |
| C27 | 0.2463 | (4) | 0.2550 | (4) | 0.1037 | (4) | | 6.4 |
| C75 | 0.7495 | (15) | 0.8026 | (15) | 0.2973 | (15) | 0.4 | 8.5 |
| C76 | 0.6263 | (20) | 0.8196 | (18) | 0.2161 | (9) | 0.4 | 11.1 |
| C77 | 0.7404 | (10) | 0.6864 | (15) | 0.2607 | (10) | 0.4 | 11.7 |
| C78 | 0.5141 | (10) | 0.7143 | (16) | 0.1993 | (10) | 0.4 | 16.8 |
| C79 | 0.5702 | (10) | 0.6219 | (9) | 0.1538 | (10) | 0.4 | 16.9 |
| C85 | 0.6973 | (10) | 0.7597 | (12) | 0.2174 | (10) | 0.6 | 19.7 |
| C86 | 0.5459 | (3) | 0.6918 | (8) | 0.1144 | (13) | 0.6 | 14.6 |
| C87 | 0.4501 | (9) | 0.6355 | (10) | 0.1778 | (16) | 0.6 | 9.7 |
| C88 | 0.5362 | (10) | 0.6626 | (9) | 0.3057 | (10) | 0.6 | 12.5 |
| O89 | 0.6579 | (10) | 0.7188 | (9) | 0.3234 | (10) | 0.6 | 14.0 |

Occupancies are 1. unless otherwise indicated

### C. Powder X-Ray Diffraction

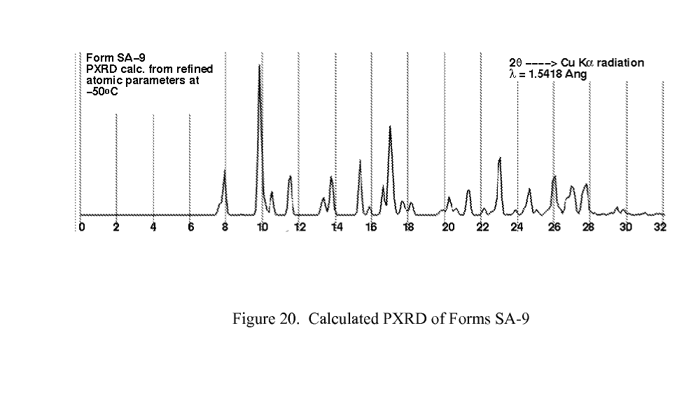

X-ray powder diffraction (PXRD) data for the SA-9 form of Compound I were obtained using the PXRD procedure described hereinabove and is shown in FIG. 20.

### Example 12

### A. Single Crystal X-Ray Measurements

Following the above Single Crystal Data procedure, the approximate unit cell dimensions in Angstroms (Å), as measured at a sample temperature of −60° C., as well as the crystalline cell volume (V), space group (sg), molecules per unit cell, and crystal density for the SC-13 form of Compound I are shown below.

Cell dimensions: a=11.104(2) Å
b=12.202(3) Å
c=13.610(3) Å
α=100.24(1)°
β=110.03(1)°
γ=109.30(1)°
Volume=1544.7(5) Å³

Space group: P-1

Molecules/unit cell (Z): 2

Density, calc g-cm⁻³: 1.302

The fractional atomic coordinates for the SC-13 form of Compound I are tabulated in Table 16 herein below.

### B. Fractional Atomic Coordinates

The arrangement of the Compound I molecules in the SC-13 form may additionally be characterized by the approximate fractional atomic coordinates listed in Table 16 below. The approximate coordinates in Table 16 will therefore vary according to the temperature at measurement. Statistical variations in these coordinates may also occur consistent with the reported error values.

TABLE 16
Fractional Atomic Parameters and Their Estimated Standard
Deviations for Form SC-13 of Compound I at −60° C.

| | | | | | | | Occu- | B |
|---|---|---|---|---|---|---|---|---|
| Atom | x | | y | | z | | pancy | (iso) |
| F2 | 0.8439 | (2) | 0.1906 | (2) | 1.00921 | (18) | | 5.8 |
| F3 | 1.0526 | (2) | 0.2007 | (2) | 1.08426 | (18) | | 6.5 |
| F6 | 0.8922 | (2) | 0.04951 | (19) | 0.93837 | (19) | | 5.8 |
| O13 | 0.4045 | (2) | 0.3178 | (2) | 0.7436 | (2) | | 4.8 |
| O22 | −0.2167 | (2) | 0.15200 | (19) | 0.3793 | (2) | | 4.4 |
| O88 | 0.3976 | (3) | 0.2356 | (3) | 1.0650 | (4) | 0.75 | 7.0 |
| O98 | 0.4552 | (3) | 0.6273 | (3) | 0.6018 | (3) | 0.75 | 5.5 |
| O99 | 0.2690 | (2) | 0.4576 | (2) | 0.6555 | (2) | 0.75 | 2.2 |
| C4 | 0.5313 | (3) | 0.1931 | (3) | 0.7521 | (3) | | 3.8 |
| C3I | 0.54285 | (7) | 0.08087 | (6) | 0.73592 | (5) | | 4.1 |
| N8 | 0.94471 | (7) | 0.34145 | (6) | 0.77992 | (5) | | 4.1 |
| N14I | 0.28105 | (7) | 0.11097 | (6) | 0.69034 | (5) | | 3.4 |
| N23I | −0.00111 | (7) | 0.31289 | (6) | 0.46181 | (5) | | 3.7 |
| N2 | 0.67618 | (7) | 0.09716 | (6) | 0.76569 | (5) | | 4.3 |
| N1 | 0.75109 | (7) | 0.22250 | (6) | 0.80341 | (5) | | 3.2 |
| C5 | 0.66835 | (7) | 0.28318 | (6) | 0.79596 | (5) | | 3.2 |
| C6I | 0.72719 | (7) | 0.41989 | (6) | 0.83442 | (5) | | 5.2 |
| C7 | 0.90072 | (7) | 0.27113 | (6) | 0.83608 | (5) | | 3.3 |
| C9I | 1.08179 | (7) | 0.38155 | (6) | 0.80002 | (5) | | 4.5 |
| C10I | 1.17450 | (7) | 0.35322 | (6) | 0.87404 | (5) | | 4.3 |
| C11I | 1.12963 | (7) | 0.28498 | (6) | 0.93450 | (5) | | 4.1 |
| C12 | 0.99037 | (7) | 0.24329 | (6) | 0.91697 | (5) | | 3.4 |
| C13 | 0.40043 | (7) | 0.21252 | (6) | 0.72748 | (5) | | 3.9 |
| C15 | 0.14375 | (7) | 0.10970 | (6) | 0.65825 | (5) | | 3.0 |
| C16 | 0.05677 | (7) | 0.05083 | (6) | 0.70482 | (5) | | 3.3 |
| C17I | −0.07976 | (7) | 0.04449 | (6) | 0.66561 | (5) | | 3.7 |
| C18I | −0.12960 | (7) | 0.09246 | (6) | 0.58431 | (5) | | 3.6 |
| C19 | −0.04185 | (7) | 0.15269 | (6) | 0.54055 | (5) | | 3.0 |
| C20I | 0.09638 | (7) | 0.16002 | (6) | 0.57935 | (5) | | 3.2 |
| C21I | 0.11125 | (7) | 0.00032 | (6) | 0.79613 | (5) | | 4.3 |
| C22 | −0.09457 | (7) | 0.20479 | (6) | 0.45338 | (5) | | 3.3 |
| C24I | −0.03275 | (7) | 0.37630 | (6) | 0.38390 | (5) | | 3.9 |
| C25I | −0.00096 | (7) | 0.35570 | (6) | 0.28647 | (5) | | 5.5 |
| C26I | 0.09200 | (7) | 0.47208 | (6) | 0.38341 | (5) | | 5.1 |
| C27 | 0.94397 | (7) | 0.17104 | (6) | 0.98578 | (5) | | 4.5 |
| C84I | 0.44642 | (7) | 0.15488 | (6) | 1.10240 | (5) | 0.75 | 10.4 |
| C85I | 0.58218 | (7) | 0.17824 | (6) | 1.09868 | (5) | 0.75 | 7.9 |
| C86I | 0.62596 | (7) | 0.29263 | (6) | 1.08082 | (5) | 0.75 | 6.2 |
| C87I | 0.50508 | (7) | 0.32327 | (6) | 1.04972 | (5) | 0.75 | 5.2 |
| C94I | 0.59710 | (7) | 0.63927 | (6) | 0.64179 | (5) | 0.75 | 6.2 |
| C95I | 0.66129 | (7) | 0.72135 | (6) | 0.58894 | (5) | 0.75 | 8.5 |
| C96I | 0.57360 | (7) | 0.78528 | (6) | 0.55255 | (5) | 0.75 | 7.9 |
| C97I | 0.44661 | (7) | 0.72215 | (6) | 0.55850 | (5) | 0.75 | 7.4 |

Occupancies are 1. unless otherwise indicated

### C. Powder X-Ray Diffraction

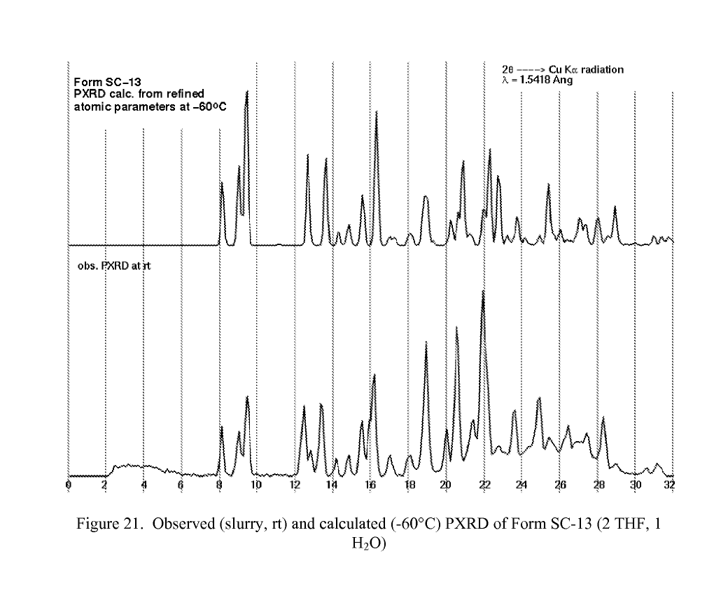

X-ray powder diffraction (PXRD) data for the SD-14 form of Compound I were obtained using the PXRD procedure described hereinabove. FIG. 21 shows observed (slurry, rt) and calculated (−60° C.) PXRD of form SC-13 (2 THF, 1H₂O).

### Example 13

### A. Single Crystal X-Ray Measurements

Following the above Single Crystal Data procedure, the approximate unit cell dimensions in Angstroms (Å), as measured at a sample temperature of −80° C., as well as the crystalline cell volume (V), space group (sg), molecules per unit cell, and crystal density for the SD-14 form of Compound I are shown below.

Cell dimensions: a=9.486(2) Å
b=9.459(1) Å
c=15.425(3) Å
α=93.98(1)°
β=95.08(1)°
γ=109.53(1)°
Volume=1292.1(4) Å³

Space group: P-1

Molecules/unit cell (Z): 2

Density, calc g-cm⁻³: 1.371

### B. Powder X-Ray Diffraction

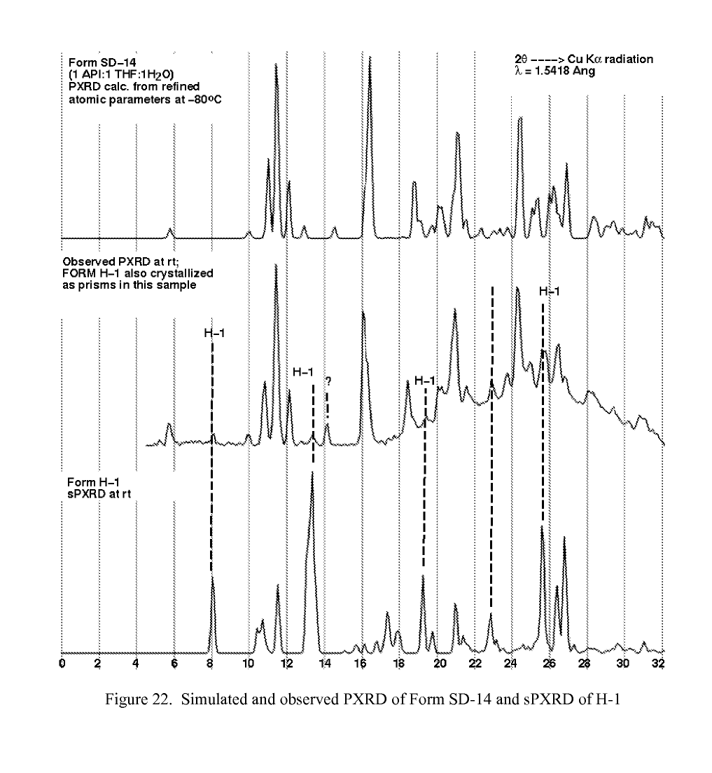

X-ray powder diffraction (PXRD) data for the SD-14 form of Compound I were obtained using the PXRD procedure described hereinabove. FIG. 22 shows simulated and observed PXRD data of form SD-14 and sPXRD of form H1.

All known lots of SD-14 (elongated plates) contain H-1 (prisms).

What is claimed is:

1. A crystalline form of the compound of Formula I:

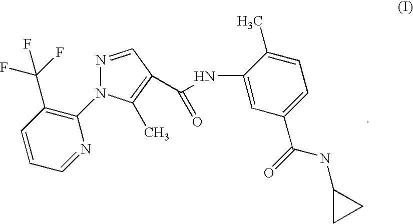

2. The crystalline form according to claim 1 comprising Form N-2.

3. The crystalline form according to claim 2, wherein said Form N-2 is in substantially pure form.

4. The crystalline form as defined in claim 2 which is characterized by unit cell parameters substantially equal to the following:
Cell dimensions from single crystal:
a=17.976(2) Å
b=12.530(1) Å
c=19.639(2) Å
α=90°
β=105.03(1)°
γ=90°
Space group: C2/c
Molecules/unit cell (Z): 8
wherein said crystalline form is at about 22° C.

5. The crystalline form as defined in claim 2 which is characterized by fractional atomic coordinates substantially as listed in Table 2.

6. The crystalline form as defined in claim 2 as characterized by a powder X-ray diffraction pattern substantially in accordance with that shown in FIG. 1.

7. The crystalline form as defined in claim 2 as characterized by a powder X-ray diffraction pattern comprising the following 2θ values (Cu Kα λ−1.5418 Å) 9.3±0.1, 11.9±0.1, 15.4±0.1, 16.9±0.1, 17.5±0.1, 18.8±0.1, 20.2±0.1 and 22.1±0.1 at about room temperature.

8. The crystalline form as defined in claim 2 which is characterized by a differential scanning calorimetry thermogram substantially in accordance with that shown in FIG. 2, having an endothermic transition with an onset in the range from about 201° C. to about 205° C.

9. The crystalline form as defined in claim 2 which is characterized by a thermal gravimetric analysis curve in accordance with that shown in FIG. 3, having a weight loss ≦0.028% at about 180° C.

10. A pharmaceutical composition comprising at least one compound according to claim 1 and a pharmaceutically-acceptable carrier or diluent.

11. A pharmaceutical composition comprising at least one compound according to claim 2 and a pharmaceutically-acceptable carrier or diluent.

12. A method of treating an inflammatory disorder comprising administering to a patient in need of such treatment a pharmaceutical composition according to claim 11.

13. The method of claim 12 in which the inflammatory disorder is selected from asthma, adult respiratory distress syndrome, chronic obstructive pulmonary disease, chronic pulmonary inflammatory disease, diabetes, inflammatory bowel disease, osteoporosis, psoriasis, graft vs. host rejection, atherosclerosis, and arthritis including rheumatoid arthritis, psoriatic arthritis, traumatic arthritis, rubella arthritis, gouty arthritis and osteoarthritis.
